SEQUENCE LISTING (42 sequences)

SEQ ID NO: 1 - protein, 701 aa, Artificial Sequence
  Synthetic construct
QVQLVQSGAEVKKPGSSVKVSCKASGYTFGNYWMQWVRQAPGQGLEWMGAIYEGTGKTVYIQKFADRVTITADKSTSTAYMELSSLRSEDTAVYYCARLSDYVSGFGYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGSGGGGSGGGGSQVQLVQSGAEVKKPGSSVKVSCKASGYPFTRYVMHWVRQAPGQCLEWMGYINPYNDGVNYNEKFKGRVTITADESTSTAYMELSSLRSEDTAVYYCARNWDTGLWGQGTTVTVSSGGGGSGGGGSGGGGSGGGGSDIQMTQSPSSLSASVGDRVTITCKASDHIGKFLTWYQQKPGKAPKLLIYGATSKLTGVPSRFSGSGSGTDFTLTISSLQPEDFATYYCQQYWSTPFTFGCGTKVEIK

SEQ ID NO: 2 - protein, 701 aa, Artificial Sequence
  Synthetic construct
QVQLVQSGAEVKKPGSSVKVSCKASGYTFGNYWMQWVRQAPGQGLEWMGAIYEGTGKTVYIQKFADRVTITADKSTSTAYMELSSLRSEDTAVYYCARLSDYVSGFGYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGSGGGGSGGGGSQVQLVQSGAEVKKPGSSVKVSCKASGYPFTRYVMHWVRQAPGQCLEWMGYINPYNDGVNYNEKFKGRVTITADESTSTAYMELSSLRSEDTAVYYCARNWDTGLWGQGTTVTVSSGGGGSGGGGSGGGGSGGGGSDIQMTQSPSSLSASVGDRVTITCKASDHIGKFLTWYQQKPGKAPKLLIYGATSKLTGVPSRFSGSGSGTDFTLTISSLQPEDFATYYCQTYWSTPFTFGCGTKVEIK

SEQ ID NO: 3 - protein, 214 aa, Artificial Sequence
  Synthetic construct
DIQMTQSPSSLSASVGDRVTITCRASKDISKYLNWYQQKPGKAPKLLIYYTSGYHSGVPSRFSGSGSGTDFTLTISSLQPEDFATYYCQQGDALPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 4 - protein, 444 aa, Artificial Sequence
  Synthetic construct
QVQLVQSGAEVKKPGSSVKVSCKASGYTFGNYWMQWVRQAPGQGLEWMGAIYEGTGKTVYIQKFADRVTITADKSTSTAYMELSSLRSEDTAVYYCARLSDYVSGFGYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSL

SEQ ID NO: 5 - protein, 119 aa, Artificial Sequence
  Synthetic construct
QVQLVQSGAEVKKPGSSVKVSCKASGYTFGNYWMQWVRQAPGQGLEWMGAIYEGTGKTVYIQKFADRVTITADKSTSTAYMELSSLRSEDTAVYYCARLSDYVSGFGYWGQGTTVTVSS

SEQ ID NO: 6 - protein, 115 aa, Artificial Sequence
  Synthetic construct
QVQLVQSGAEVKKPGSSVKVSCKASGYPFTRYVMHWVRQAPGQCLEWMGYINPYNDGVNYNEKFKGRVTITADESTSTAYMELSSLRSEDTAVYYCARNWDTGLWGQGTTVTVSS

SEQ ID NO: 7 - protein, 107 aa, Artificial Sequence
  Synthetic construct
DIQMTQSPSSLSASVGDRVTITCRASKDISKYLNWYQQKPGKAPKLLIYYTSGYHSGVPSRFSGSGSGTDFTLTISSLQPEDFATYYCQQGDALPPTFGGGTKVEIK

SEQ ID NO: 8 - protein, 107 aa, Artificial Sequence
  Synthetic construct
DIQMTQSPSSLSASVGDRVTITCKASDHIGKFLTWYQQKPGKAPKLLIYGATSKLTGVPSRFSGSGSGTDFTLTISSLQPEDFATYYCQQYWSTPFTFGCGTKVEIK

SEQ ID NO: 9 - protein, 107 aa, Artificial Sequence
  Synthetic construct
DIQMTQSPSSLSASVGDRVTITCKASDHIGKFLTWYQQKPGKAPKLLIYGATSKLTGVPSRFSGSGSGTDFTLTISSLQPEDFATYYCQTYWSTPFTFGCGTKVEIK

SEQ ID NO: 10 - protein, 13 aa, Artificial Sequence
  Synthetic construct
KASGYTFGNYWMQ

SEQ ID NO: 11 - protein, 17 aa, Artificial Sequence
  Synthetic construct
AIYEGTGKTVYIQKFAD

SEQ ID NO: 12 - protein, 12 aa, Artificial Sequence
  Synthetic construct
ARLSDYVSGFGY

SEQ ID NO: 13 - protein, 13 aa, Artificial Sequence
  Synthetic construct
KASGYPFTRYVMH

SEQ ID NO: 14 - protein, 17 aa, Artificial Sequence
  Synthetic construct
YINPYNDGVNYNEKFKG

SEQ ID NO: 15 - protein, 8 aa, Artificial Sequence
  Synthetic construct
ARNWDTGL

SEQ ID NO: 16 - protein, 11 aa, Artificial Sequence
  Synthetic construct
RASKDISKYLN

SEQ ID NO: 17 - protein, 8 aa, Artificial Sequence
  Synthetic construct
YYTSGYHS

SEQ ID NO: 18 - protein, 9 aa, Artificial Sequence
  Synthetic construct
QQGDALPPT

SEQ ID NO: 19 - protein, 11 aa, Artificial Sequence
  Synthetic construct
KASDHIGKFLT

SEQ ID NO: 20 - protein, 8 aa, Artificial Sequence
  Synthetic construct
YGATSKLT

SEQ ID NO: 21 - protein, 9 aa, Artificial Sequence
  Synthetic construct
QQYWSTPFT

SEQ ID NO: 22 - protein, 9 aa, Artificial Sequence
  Synthetic construct
QTYWSTPFT

SEQ ID NO: 23 - protein, 15 aa, Artificial Sequence
  Synthetic construct
GGGGSGGGGSGGGGS

SEQ ID NO: 24 - protein, 20 aa, Artificial Sequence
  Synthetic construct
GGGGSGGGGSGGGGSGGGGS

SEQ ID NO: 25 - protein, 22 aa, Artificial Sequence
  Synthetic construct
QVQLVQSGAEVKKPGSSVKVSC

SEQ ID NO: 26 - protein, 14 aa, Artificial Sequence
  Synthetic construct
WVRQAPGQGLEWMG

SEQ ID NO: 27 - protein, 30 aa, Artificial Sequence
  Synthetic construct
RVTITADKSTSTAYMELSSLRSEDTAVYYC

SEQ ID NO: 28 - protein, 11 aa, Artificial Sequence
  Synthetic construct
WGQGTTVTVSS

SEQ ID NO: 29 - protein, 22 aa, Artificial Sequence
  Synthetic construct
QVQLVQSGAEVKKPGSSVKVSC

SEQ ID NO: 30 - protein, 14 aa, Artificial Sequence
  Synthetic construct
WVRQAPGQCLEWMG

SEQ ID NO: 31 - protein, 30 aa, Artificial Sequence
  Synthetic construct
RVTITADESTSTAYMELSSLRSEDTAVYYC

SEQ ID NO: 32 - protein, 11 aa, Artificial Sequence
  Synthetic construct
WGQGTTVTVSS

SEQ ID NO: 33 - protein, 23 aa, Artificial Sequence
  Synthetic construct
DIQMTQSPSSLSASVGDRVTITC

SEQ ID NO: 34 - protein, 14 aa, Artificial Sequence
  Synthetic construct
WYQQKPGKAPKLLI

SEQ ID NO: 35 - protein, 32 aa, Artificial Sequence
  Synthetic construct
GVPSRFSGSGSGTDFTLTISSLQPEDFATYYC

SEQ ID NO: 36 - protein, 10 aa, Artificial Sequence
  Synthetic construct
FGCGTKVEIK

SEQ ID NO: 37 - protein, 23 aa, Artificial Sequence
  Synthetic construct
DIQMTQSPSSLSASVGDRVTITC

SEQ ID NO: 38 - protein, 14 aa, Artificial Sequence
  Synthetic construct
WYQQKPGKAPKLLI

SEQ ID NO: 39 - protein, 32 aa, Artificial Sequence; identical to SEQ ID NO: 35
  Synthetic construct

SEQ ID NO: 40 - protein, 10 aa, Artificial Sequence
  Synthetic construct
FGGGTKVEIK

SEQ ID NO: 41 - protein, 325 aa, Artificial Sequence
  Synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSL

SEQ ID NO: 42 - protein, 107 aa, Artificial Sequence [sequence omitted]

(19) United States
(12) Patent Application Publication
(10) Pub. No.: US 2018/0291093 A1
(43) Pub. Date: Oct. 11, 2018

(54) ANTI-CGRP/ANTI-IL-23 BISPECIFIC ANTIBODIES AND USES THEREOF

(71) Applicant: Eli Lilly and Company, Indianapolis, IN (US)

(72) Inventors: Robert Jan Benschop, Indianapolis, IN (US); Rohn L Millican, JR., Indianapolis, IN (US); Barrett Allan, Encinitas, CA (US); Catherine Brautigam Beidler, Poway, CA (US)

(73) Assignee: Eli Lilly and Company, Indianapolis, IN (US)

(21) Appl. No.: 15/764,368

(22) PCT Filed: Oct. 30, 2015
(86) PCT No.: PCT/US2015/058362

§ 371 Date: Mar. 29, 2018

(51) Int. Cl. C07K 16/24; C07K 16/18

(52) U.S. Cl. CPC C07K 16/244; A61K 2039/505; C07K 16/18

(57) ABSTRACT

Bispecific antibodies are provided that bind Calcitonin Gene Related Peptide (CGRP) and Interleukin-23 (IL-23) and are characterized as having high affinity and strong simultaneous neutralizing properties to both CGRP and IL-23. The bispecific antibodies of the invention are useful for treating various autoimmune diseases including Inflammatory Bowel Disease, such as Crohn's Disease and Ulcerative Colitis, Psoriatic Arthritis (PsA) and ankylosing spondylitis (AS).

The present invention is in the field of medicine, particularly in the novel field of bispecific antibodies directed against Calcitonin Gene Related Peptide (CGRP) and Interleukin-23 (IL-23). The bispecific antibodies of the present invention are expected to be useful in treating autoimmune diseases including Inflammatory Bowel Disease (IBD), such as Crohn's Disease (CD) and Ulcerative Colitis (UC), Psoriatic Arthritis (PsA) and ankylosing spondylitis (AS).

Autoimmune diseases arise from the body's production of an immune response against its own tissue. Autoimmune diseases are often chronic and can be debilitating and even life-threatening. IBD, which generically represents a group of disorders such as CD and UC, is a common chronic relapsing autoimmune disease characterized pathologically by intestinal inflammation and epithelial injury. Other forms of chronic autoimmune diseases, such PsA and AS, may affect the axial and/or peripheral skeleton.

Interleukin 23 (IL-23) is a heterodimeric cytokine believed to be important in the activation of a range of inflammatory cells required for the induction of chronic inflammation. IL-23, which is believed to be an upstream regulator of IL-6, IL-17, GM-CSF and IL-22 secretion, is composed of a p19 subunit (IL23p19) covalently paired to a p40 subunit (the p40 subunit is also shared with cytokine IL-12). Additionally, IL-23 has been implicated as playing an important role in memory/pathogenic T-cell inflammatory response as well as playing a role in the regulation of innate lymphoid cell inflammatory activity. There is evidence that IL-23 regulation of the cytokines IL-6, IL-17, GM-CSF and IL-22 is associated with inflammatory diseases including IBD and other autoimmune diseases.

CGRP is a 37 amino acid neuropeptide secreted by the nerves of the central and peripheral nervous systems. CGRP is widely distributed in sensory nerves, both in the peripheral and central nervous system and displays a large number of different biological activities. For instance, it is a potent vasodilator with microvasculature being sensitive thereto. When released from trigeminal and other nerve fibers, CGRP is thought to mediate its biological responses by binding to specific cell surface receptors. CGRP is believed to play a role in the modulation and/or transmission of pain signaling and in neurogenic inflammation. CGRP has been reported to play a role in migraines as CGRP is released upon stimulation of sensory nerves. The release of CGRP increases vascular permeability and subsequent plasma protein leakage (plasma protein extravasation) in tissues innervated by trigeminal nerve fibers upon stimulation of these fibers. In addition, studies have reported that infusion of CGRP in patients who suffer from migraines has resulted in migraine-like symptoms.

Current FDA approved treatments for autoimmune diseases such as IBD include corticosteroids, often used to treat acute inflammation, and bioproducts, many of which (such as REMICADE®, ENBREL® and HUMIRA) attempt to target and neutralize TNFα in the body. Another bioproduct approved for treatment of PsA includes STELARA® which attempts to target the shared p40 subunit of cytokines IL-12 and IL-23. Current treatments have demonstrated efficacy for reducing symptoms and slowing progression of some autoimmune diseases in a subset of patients. However, a large percentage of patients are nonresponsive to currently available treatments (for example, induction of remission occurs in only 30-50% of CD patients treated with TNFα neutralization, and loss of response to TNFα neutralization occurs in between 23 and 46% of patients following 12 months of treatment). Alternative therapies for autoimmune diseases include antibodies that bind to the p19 subunit of IL-23, such as those disclosed in U.S. Pat. No. 9,023,358.

While currently approved treatments for autoimmune diseases treat the inflammatory aspect of the disease, said treatments have proved ineffective in treating associated pain. Even in patients suffering from IBD (CD and UC) that are responsive to anti-TNFα therapy, pain remains. It is thought that inflammation associated with autoimmune diseases drives central sensitization to pain leading to hyperalgesia and allodynia. The consequence is that pain can be present even after inflammation has subsided with a high percentage of patients continuing to take pain medication. The standard therapies for pain in patients suffering from IBD are analgesics including NSAIDS, COX-2 inhibitors and opiates. At present, patients suffering from IBD are filling a similar number of analgesic prescriptions both prior to and post the introduction of biologic therapy. Antibodies that bind to CGRP, such as those described in U.S. Pat. No. 9,073,991, have been suggested as therapeutics for migraine.

One approach to such alternative therapies may include the co-administration of two different bioproducts (e.g., antibodies) treating different aspects of the autoimmune disease (e.g. pathology of the disease and associated pain). Co-administration requires either injections of two separate products or a single injection of a co-formulation of two different antibodies. While two injections permit flexibility of dose amounts and timing, it is inconvenient to patients both for compliance and pain. Moreover, while a co-formulation might provide some flexibility of dose amounts, it is often quite challenging or impossible to find formulation conditions having acceptable viscosity (at relatively high concentration) and that promote chemical and physical stability of both antibodies due to different molecular characteristics of the two antibodies. Additionally, co-administration and co-formulation involve the additive costs of two different drug therapies which can increase patient and/or payer costs.

Thus, there remains a need for alternative therapies for treatment of autoimmune diseases that have both disease modification and pain management properties and preferably such alternative therapies comprise a bispecific antibody.

The present invention provides a bispecific antibody comprising an immunoglobulin G antibody (IgG) and two single chain variable fragments (scFv).

More specifically, the present invention provides a bispecific antibody comprising an IgG and two scFv wherein,
(a) said IgG comprises two heavy chains (HC) and two light chains (LC), each HC comprises a heavy chain variable region (HCVR1) comprising heavy chain CDRs (HCDR) 1-3 and each light chain comprises a light chain variable region (LCVR1) comprising light chain CDRs (LCDR) 1-3, wherein the amino acid sequence of HCDR1 is SEQ ID NO: 10, the amino acid sequence of HCDR2 is SEQ ID NO: 11, the amino acid sequence of HCDR3 is SEQ ID NO: 12, the amino acid sequence of LCDR1 is SEQ ID NO: 16, the amino acid sequence of LCDR2 is SEQ ID NO: 17, and the amino acid sequence of LCDR3 is SEQ ID NO: 18; and
(b) each scFv comprises a heavy chain variable region (HCVR2) and a light chain variable region (LCVR2), the HCVR2 comprising HCDRs 4-6, and the LCVR2 comprising LCDRs 4-6, wherein the amino acid sequence of HCDR4 is SEQ ID NO: 13, the amino acid sequence of HCDR5 is SEQ ID NO: 14, the amino acid sequence of HCDR6 is SEQ ID NO: 15, the amino acid sequence of LCDR4 is SEQ ID NO: 19, the amino acid sequence of LCDR5 is SEQ ID NO: 20, and the amino acid sequence of LCDR6 is SEQ ID NO: 21 or SEQ ID NO: 22,

wherein each scFv is linked at the N-terminus of HCVR2 of each scFv to said IgG antibody at the C-terminus of each IgG HC via a polypeptide linker (L1),

and wherein the HCVR2 of each scFv is linked at the C-terminus of the HCVR2 to the LCVR2 of the same scFv at the N-terminus of the LCVR2 of the same scFv via a second polypeptide linker (L2).

The bispecific antibody of the present invention binds to CGRP and the p19 subunit of IL-23.

Preferably, the amino acid sequence of LCDR6 is SEQ ID NO: 21.

Further preferably, the amino acid sequence of LCDR6 is SEQ ID NO: 22.

In a further embodiment of the bispecific antibody of the present invention, the amino acid sequence of HCVR1 of each HC is SEQ ID NO: 5, the amino acid sequence of LCVR1 of each LC is SEQ ID NO: 7, the amino acid sequence of HCVR2 of each scFv is SEQ ID NO: 6 and the amino acid sequence of LCVR2 of each scFv is SEQ ID NO: 8 or SEQ ID NO: 9.

Preferably, the amino acid sequence of LCVR2 of each scFv is SEQ ID NO: 8.

Further preferably, the amino acid sequence of LCVR2 of each scFv is SEQ ID NO: 9.

In a still further embodiment of the bispecific antibody of the present invention, the amino acid sequence of each HC is SEQ ID NO: 4, the amino acid sequence of each LC is SEQ ID NO: 3, the amino acid sequence of HCVR2 of each scFv is SEQ ID NO: 6 and the amino acid sequence of LCVR2 of each scFv is SEQ ID NO: 8 or SEQ ID NO: 9.

Preferably, the amino acid sequence of LCVR2 of each scFv is SEQ ID NO: 8.

Further preferably, the amino acid sequence of LCVR2 of each scFv is SEQ ID NO: 9.

In a preferred aspect of the above embodiments of the present invention, the amino acid sequence of L1 is SEQ ID NO: 23 and the amino acid sequence of L2 is SEQ ID NO: 24.

In a preferred embodiment of the bispecific antibody of the present invention, the amino acid sequence of each HC is SEQ ID NO: 4, the amino acid sequence of each LC is SEQ ID NO: 3, the amino acid sequence of HCVR2 of each scFv is SEQ ID NO: 6, the amino acid sequence of LCVR2 of each scFv is SEQ ID NO: 8, the amino acid sequence of L1 is SEQ ID NO: 23 and the amino acid sequence of L2 is SEQ ID NO: 24.

In a further preferred embodiment of the bispecific antibody of the present invention, the amino acid sequence of each HC is SEQ ID NO: 4, the amino acid sequence of each LC is SEQ ID NO: 3, the amino acid sequence of HCVR2 of each scFv is SEQ ID NO: 6, the amino acid sequence of LCVR2 of each scFv is SEQ ID NO: 9, the amino acid sequence of L1 is SEQ ID NO: 23 and the amino acid sequence of L2 is SEQ ID NO: 24.

Significant problems associated with chemical and physical stability were addressed when building a bispecific antibody of the present invention. Many changes were required in the starting bispecific antibody to sufficiently overcome a myriad of issues that can be associated with bispecific antibodies, such as expressing a physically stable molecule, stabilizing the VH/VL interface of the single chain fragment variable region (scFv), increasing thermal and salt-dependent stability, decreasing aggregation, increasing solubility at high concentrations, and/or rebalancing the electrostatic distribution in the binding surfaces of the bispecific antibody, all while maintaining binding affinity for both targeted antigens; CGRP and the p19 subunit of IL-23.

Bispecific antibodies of the present invention are thermally stable and physically stable. Moreover, bispecific antibodies of the present invention may also exhibit low aggregation. Furthermore, bispecific antibodies of the present invention may also neutralize human CGRP and human IL23p19 (the p19 subunit of IL-23), as well as simultaneously binding both ligands. The presently claimed antibodies may also avoid the challenges of finding formulation conditions that must satisfy the different molecular characteristics of two different, separate antibodies.

The IgG part of a first bispecific antibody of the present invention comprises two identical heavy chains (IgG HC)(SEQ ID NO: 4).

Each IgG HC is attached at its C-terminus via a first polypeptide linker (L1)(SEQ ID NO: 23) to an identical scFv portion that specifically binds to the p19 subunit of IL-23.

Each heavy chain scFv portion (HCVR2)(SEQ ID NO: 6) is attached at its C-terminus via a second polypeptide linker (L2)(SEQ ID NO: 24) to a scFv light chain (LCVR2)(SEQ ID NO: 8).

The complete linear amino acid sequence of each identical heavy chain part of the first bispecific antibody of the invention, starting from the N-terminal residue of the IgG₄ HC and ending at the C-terminal residue of the scFv LC is provided in SEQ ID NO: 1.

Similarly, the complete amino acid sequence of each identical LC of the first bispecific antibody of the invention, starting from the N-terminal residue of the variable domain and ending at the C-terminal residue of the LC kappa constant region is provided in SEQ ID NO: 3.

The relationship of the various regions and linkers of an exemplified first bispecific antibody of the present invention is as follows (numbering of amino acids applies linear numbering; assignment of amino acids to variable domains is based on the International Immunogenetics Information System® available at www.imgt.org; assignment of amino acids to CDR domains is based on the well-known Kabat (Kabat et al., Ann. NY Acad. Sci. 190:382-93 (1971); Kabat et al., Sequences of Proteins of Immunological Interest, Fifth Edition, U.S. Department of Health and Human Services, NIH Publication No. 91-3242 (1991)) and North (North et al., A New Clustering of Antibody CDR Loop Conformations, Journal of Molecular Biology, 406:228-256 (2011)) numbering conventions as reflected in Tables 1(a)-(c)):

TABLE 1(a)
Amino acid regions of Bispecific Antibody 1-IgG HC-L1-scFv HCVR2-
L2-scFv LCVR2
SEQ ID NO: 1

| | Region | Positions |
|---|---|---|
| HCVR1 CGRP | FRH1-1 (SEQ | 1-22 |
| (SEQ ID NO: 5) | ID NO: 25) | QVQLVQSGAEVKKPGSSVKVSC |
| | HCDR1 (SEQ | 23-35 |
| | ID NO: 10) | KASGYTFGNYWMQ |
| | FRH1-2 (SEQ | 36-49 |
| | ID NO: 26) | WVRQAPGQGLEWMG |
| | HCDR2 (SEQ | 50-66 |
| | ID NO: 11) | AIYEGTGKTVYIQKFAD |
| | FRH1-3 (SEQ | 67-96 |
| | ID NO: 27) | RVTITADKSTSTAYMELSSLRSEDTAVYYC |
| | HCDR3 (SEQ | 97-108 |
| | ID NO: 12) | ARLSDYVSGFGY |
| | FRH1-4 (SEQ | 109-119 |
| | ID NO: 28) | WGQGTTVTVSS |
| Constant | CH (SEQ ID | 120-444 |
| | NO: 41) | ASTKGPSVFPLAPCSRSTSESTAALGCLVKDY |
| | | FPEPVTVSWNSGALTSGVHTFPAVLQSSGLYS |
| | | LSSVVTVPSSSLGTKTYTCNVDHKPSNTKVDK |
| | | RVESKYGPPCPPCPAPEAAGGPSVFLFPPKPK |
| | | DTLMISRTPEVTCVVVDVSQEDPEVQFNWYV |
| | | DGVEVHNAKTKPREEQFNSTYRVVSVLTVLH |
| | | QDWLNGKEYKCKVSNKGLPSSIEKTISKAKG |
| | | QPREPQVYTLPPSQEEMTKNQVSLTCLVKGF |
| | | YPSDIAVEWESNGQPENNYKTTPPVLDSDGSF |
| | | FLYSRLTVDKSRWQEGNVFSCSVMHEALHNH |
| | | YTQKSLSLSL |
| Linker | L1 (SEQ ID | 445-459 |
| | NO: 23) | GGGGSGGGGSGGGGS |
| HCVR2 IL-23 | FRH2-1 (SEQ | 460-481 |
| (SEQ ID NO: 6) | ID NO: 29) | QVQLVQSGAEVKKPGSSVKVSC |
| | HCDR4 (SEQ | 482-494 |
| | ID NO: 13) | KASGYPFTRYVMH |
| | FRH2-2 (SEQ | 495-508 |
| | ID NO: 30) | WVRQAPGQCLEWMG |
| | HCDR5 (SEQ | 509-525 |
| | ID NO: 14) | YINPYNDGVNYNEKFKG |
| | FRH2-3 (SEQ | 526-555 |
| | ID NO: 31) | RVTITADESTSTAYMELSSLRSEDTAVYYC |
| | HCDR6 (SEQ | 556-563 |
| | ID NO: 15) | ARNWDTGL |
| | FRH2-4 (SEQ | 564-574 |
| | ID NO: 32) | WGQGTTVTVSS |
| Linker | L2 (SEQ ID | 575-594 |
| | NO: 24) | GGGGSGGGGSGGGGSGGGGS |
| LCVR2 IL-23 | FRL2-1 (SEQ | 595-617 |
| (SEQ ID NO: 8) | ID NO: 33) | DIQMTQSPSSLSASVGDRVTITC |
| | LCDR4 | (SEQ 618-628 |
| | ID NO: 19) | KASDHIGKFLT |
| | FRL2-2 (SEQ | 629-642 |
| | ID NO: 34) | WYQQKPGKAPKLLI |
| | LCDR5 (SEQ | 643-650 |
| | ID NO: 20) | YGATSKLT |
| | FRL2-3 (SEQ | 651-682 |
| | ID NO: 35) | GVPSRFSGSGSGTDFTLTISSLQPEDFATYYC |
| | LCDR6 | (SEQ 683-691 |
| | ID NO: 21) | QQYWSTPFT |
| | FRL2-4 (SEQ | 692-701 |
| | ID NO: 36) | FGCGTKVEIK |

TABLE 1(b)
Amino acid regions of Bispecific Antibody 1-IgG LC
SEQ ID NO: 3

| | Regions | Positions |
|---|---|---|
| LCVR1 CGRP | FRL1-1 (SEQ | 1-23 |
| (SEQ ID NO: 7) | ID NO: 37) | DIQMTQSPSSLSASVGDRVTITC |
| | LCDR1 (SEQ | 24-34 |
| | ID NO: 16) | RASKDISKYLN |
| | FRL1-2 (SEQ | 35-48 |
| | ID NO: 38) | WYQQKPGKAPKLLI |
| | LCDR2 (SEQ | 49-56 |
| | ID NO: 17) | YYTSGYHS |
| | FRL1-3 (SEQ | 57-88 |
| | ID NO: 39) | GVPSRFSGSGSGTDFTLTISSLQPEDFATYYC |
| | LCDR3 (SEQ | 89-97 |
| | ID NO: 18) | QQGDALPPT |
| | FRL1-4 (SEQ | 98-107 |
| | ID NO: 40) | FGGGTKVEIK |
| Constant | CL (SEQ ID | 108-214 |
| | NO: 42) | RTVAAPSVFIFPPSDEQLKSGTASVVCLLNNF |
| | | YPREAKVQWKVDNALQSGNSQESVTEQDSK |
| | | DSTYSLSSTLTLSKADYEKHKVYACEVTHQG |
| | | LSSPVTKSFNRGEC |

According to a second exemplified bispecific antibody of the present invention, LCDR6 incorporates an engineered single amino acid change that substitutes threonine (T) for glutamine (Q) at position 684 (Q684T) in SEQ ID NO: 1 such that the LCDR6 of the second exemplified bispecific antibody of the present invention has the following sequence QTYWSTPFT (SEQ ID NO: 22). No additional changes were made and, consequently, all remaining amino acid sequences of the second exemplified bispecific antibody of the present invention are identical to those of the first exemplified bispecific antibody described above.

The complete linear amino acid sequence of each identical heavy chain part of the second bispecific antibody of the invention, which comprises LCDR6 having SEQ ID NO: 22, starting from the N-terminal residue of the IgG₄ HC and ending at the C-terminal residue of the scFv LC is provided in SEQ ID NO: 2.

Similarly, the complete amino acid sequence of each identical LC of the second bispecific antibody of the invention, starting from the N-terminal residue of the LC variable domain and ending at the C-terminal residue of the LC constant region is provided in SEQ ID NO: 3.

The present invention further provides a bispecific antibody wherein each of the HCs form an inter-chain disulfide bond with each of the LCs; wherein each of the HCs forms an inter-chain disulfide bond with the other HC; and wherein each of the scFvs forms an intra-chain disulfide bond between HCVR2 and LCVR2. According to the exemplified bispecific antibody of the present invention presented in Tables 1(a) and (b), an inter-chain disulfide bond of each of the HCs and each of the LCs forms between cysteine residue 133 (of SEQ ID NO: 1 and SEQ ID NO: 2) of the HC, and cysteine residue 214 (of SEQ ID NO: 3) of the LC; at least two inter-chain disulfide bonds form between the two HCs, the first inter-chain disulfide bond forming between cysteine residue 225 (of SEQ ID NO: 1 or SEQ ID NO: 2) of the HC and cysteine residue 225 (of SEQ ID NO: 1 or SEQ ID NO: 2) of the other HC, the second inter-chain disulfide bond forming between cysteine residue 228 (of SEQ ID NO: 1 or SEQ ID NO: 2) of the HC and cysteine residue 228 (of SEQ ID NO: 1 or SEQ ID NO: 2) of the other HC; and an intra-chain disulfide bond of the scFv is formed between cysteine residue 503 (of SEQ ID NO: 1 or SEQ ID NO: 2) of the HCVR2 and cysteine residue 694 (of SEQ ID NO:1 or SEQ ID NO: 2) of the LCVR2.

According to some embodiments of the present invention, a bispecific antibody comprising glycosylation of the HC is provided. According to the exemplified bispecific antibody of the present invention presented in Tables 1(a) and (b), glycosylation of the HC occurs at the asparagine residue 296 of SEQ ID NO: 1 or SEQ ID NO: 2.

Given the amino acid sequences provided herein, one of ordinary skill in the art could use this knowledge to design a DNA molecule to encode and express any bispecific antibody, or fragment thereof, described hereinabove. The present invention thus encompasses all DNA sequences encoding a bispecific antibody or fragment thereof according to the invention.

In particular, the present invention provides a DNA molecule comprising a polynucleotide sequence encoding a polypeptide chain comprising a HC, a scFv, a first polypeptide linker L1 and a second polypeptide linker L2 of the bispecific antibody of present invention.

According to an embodiment of the present invention, the amino acid sequence of the encoded polypeptide chain is SEQ ID NO: 1.

According to an alternative embodiment of the present invention, the amino acid sequence of the encoded polypeptide is SEQ ID NO: 2.

The present invention also provides an expression vector comprising a polynucleotide sequence encoding the polypeptide of SEQ ID NO: 1 and a polynucleotide sequence encoding the polypeptide of SEQ ID NO: 3.

The present invention also provides an expression vector comprising a polynucleotide sequence encoding the polypeptide of SEQ ID NO: 2 and a polynucleotide sequence encoding the polypeptide of SEQ ID NO: 3.

The present invention also provides a recombinant host cell comprising a DNA molecule comprising a polynucleotide sequence encoding a polypeptide chain comprising a HC, a scFv, a first polypeptide linker L1 and a second polypeptide linker L2 of the bispecific antibody of present invention, wherein the amino acid sequence of the polypeptide chain is SEQ ID NO: 1 or SEQ ID NO: 2, and a DNA molecule comprising a polynucleotide sequence encoding a polypeptide chain comprising a LC of the bispecific antibody the present invention, wherein the amino acid sequence of the LC is SEQ ID NO: 3, wherein the cell is capable of expressing a bispecific antibody of the present invention, said bispecific antibody comprising an IgG that binds CGRP conjugated to two scFvs that bind IL23p19.

The present invention also provides a recombinant host cell transformed with a DNA molecule comprising a polynucleotide sequence encoding a polypeptide chain comprising a HC, a scFv, a first polypeptide linker L1 and a second polypeptide linker L2 of the bispecific antibody of present invention, wherein the amino acid sequence of the polypeptide chain is SEQ ID NO: 1 or SEQ ID NO: 2, and a DNA molecule comprising a polynucleotide sequence encoding a polypeptide chain comprising a LC of the bispecific antibody the present invention, wherein the amino acid sequence of the LC is SEQ ID NO: 3, said bispecific antibody comprising an IgG that binds CGRP conjugated to two scFvs that bind IL23p19.

The present invention also provides a process for producing a bispecific antibody of the present invention, the process comprising cultivating a recombinant host cell of the present invention under conditions such that the bispecific antibody is expressed, and recovering the expressed bispecific antibody.

The present invention also provides a bispecific antibody according to the present invention produced by said process.

Preferably, the recombinant host cells is a mammalian host cell selected from the group consisting of CHO, NS0, HEK293 and COS cells.

The present invention also provides a method of treating autoimmune diseases comprising administering to a patient in need thereof an effective amount of a bispecific antibody of the present invention.

The present invention also provides a method of treating IBD, such as CD and/or UC, comprising administering to a patient in need thereof an effective amount of a bispecific antibody of the present invention.

The present invention also provides a method of treating PsA and/or ankylosing spondylitis comprising administering to a patient in need thereof an effective amount of a bispecific antibody of the present invention.

The present invention also provides a bispecific antibody of the present invention for use in therapy.

The present invention also provides a bispecific antibody of the present invention for use in the treatment of autoimmune diseases including IBD, such as CD and/or UC.

The present invention also provides a bispecific antibody of the present invention for use in the treatment of autoimmune diseases including PsA and/or ankylosing spondylitis.

The present invention also provides a pharmaceutical composition comprising a bispecific antibody of the present invention and one or more pharmaceutically acceptable carriers, diluents or excipients.

Another embodiment of the present invention comprises use of a bispecific antibody of the present invention in the manufacture of a medicament for the treatment of ulcerative colitis and/or Crohn's disease.

An additional embodiment of the present invention comprises use of a bispecific antibody of the present invention in the manufacture of a medicament for the treatment of psoriatic arthritis and/or ankylosing spondylitis.

### DEFINITIONS

When used herein the term “bispecific antibody” refers to a molecule comprising an immunoglobulin G antibody (IgG) conjugated to two single chain variable fragments (scFv). As referred to herein, a bispecific antibody of the present invention comprises an IgG and two scFv's, wherein each scFv is linked at the N-terminus of HCVR2 of each scFv to said IgG antibody at the C-terminus of each IgG HC via a polypeptide linker (L1) and wherein the HCVR2 of each scFv is linked at the C-terminus of the HCVR2 to the LCVR2 of the same scFv at the N-terminus of the LCVR2 of the same scFv via a second polypeptide linker (L2). The IgG and scFvs of a bispecific antibody of the present invention specifically bind different antigens (CGRP and the p19 subunit of IL-23, respectively). Notably, the bispecific antibody of the present invention binds to the p19 subunit of IL-23 but does not bind to the p40 subunit of IL-23 that is shared with IL-12.

As referred to herein, the term “single chain variable fragment” (scFv), refers to a polypeptide chain comprising a heavy chain variable region (HCVR2) and a light chain variable region (LCVR2) connected via a polypeptide linker (L2). Additionally, as referred to herein (and as represented in the following schematic), the HCVR2 of each scFv is: a) linked, at its N-terminus, to the C-terminus of one HC of the IgG via a polypeptide linker (L1); and b) L1 is linked, at its C-terminus, to the N-terminus of the LCVR2 of the same scFv via a second polypeptide linker (L2). Further, each scFv of the present invention includes a disulfide bond formed between a cysteine residue of HCVR2 and a cysteine residue of LCVR2 of the same polypeptide chain (as represented in the following schematic):

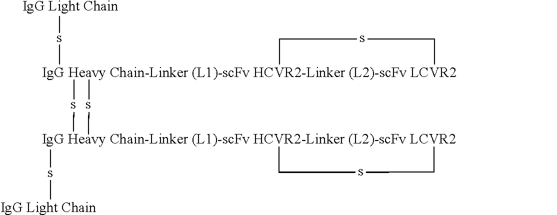

A “parent antibody” or “parental antibody,” as used interchangeably herein, is an antibody encoded by an amino acid sequence which is used in the preparation of one of the IgG and scFv of the bispecific antibody, for example through amino acid substitutions and structural alteration. The parent antibody may be a murine, chimeric, humanized or human antibody.

The terms “Kabat numbering” or “Kabat labeling” are used interchangeably herein. These terms, which are recognized in the art, refer to a system of numbering amino acid residues which are more variable (i.e., hypervariable) than other amino acid residues in the heavy and light chains variable regions of an antibody (Kabat, et al., Ann. NY Acad. Sci. 190:382-93 (1971); Kabat et al., Sequences of Proteins of Immunological Interest, Fifth Edition, U.S. Department of Health and Human Services, NIH Publication No. 91-3242 (1991)).

The terms “North numbering” or “North labeling” are used interchangeably herein. These terms, which are recognized in the art, refer to a system of numbering amino acid residues which are more variable (i.e., hypervariable) than other amino acid residues in the heavy and light chains variable regions of an antibody and is based, at least in part, on affinity propagation clustering with a large number of crystal structures, as described in (North et al., A New Clustering of Antibody CDR Loop Conformations, Journal of Molecular Biology, 406:228-256 (2011).

The terms “patient,” “subject,” and “individual,” used interchangeably herein, refer to an animal, preferably the term refers to humans. In certain embodiments, the subject, preferably a human, is further characterized with a disease or disorder or condition (e.g., an autoimmune disorder) that would benefit from a decreased level or decreased bioactivity of both IL-23 and CGRP. In another embodiment the subject, preferably a human, is further characterized as being at risk of developing a disorder, disease or condition that would benefit from a decreased level or decreased bioactivity of both IL-23 and CGRP.

### Bispecific Antibody Engineering

Significant problems associated with chemical and physical stability were encountered when constructing a bispecific antibody of the present invention. Problems encountered included poor to no expression, poor purification recovery, low thermostability, high salt-dependent aggregation, diabody formation (and challenges in reducing diabodies through purification), high solution viscosity, low binding affinity and cross-reactivity.

For example, initial attempts in constructing an IgG-scFv formatted bispecific antibody included constructs in which a parental IL-23 antibody (the IL-23 antibody described in U.S. Pat. No. 9,023,358) comprised the IgG antibody portion and a parental CGRP antibody (see for example U.S. Pat. No. 9,073,991) comprised the scFv portion of the bispecific antibody. Other initially attempted constructs included the parental IL-23 antibody comprising the scFv portion while the CGRP antibody comprised the IgG portion of the bispecific antibody. Additionally, initial constructs included the scFv portion being conjugated to the IgG portion in various configurations, including at the amino-terminus or the carboxyl terminus for both the heavy and light chains, respectively. Moreover, initial constructs included the scFv portion varying in arrangement of the HCVR2 and LCVR2 (e.g., IgG portion (C or N terminus)-linker 1-LCVR2 or HCVR2-linker 2- the other of LCVR2 or HCVR2). Further, parental IL-23 antibody constructs included combinations of heavy chain germline frameworks VH 5-51 and 1-69, and light chain germline frameworks VK 02, VK 12 and VK B3. Parental CGRP antibody constructs (when comprising the IgG portion) included an IgG₄ subclass structure having three amino acid mutations (from native IgG₄) within the constant region (CH). Initial constructs were cloned into a human IgG4-Fc mammalian expression vector. However, initially produced bispecific constructs as (described above) exhibited one or more chemical and/or physical problem(s) described above. For instance, constructs wherein the scFv portion is positioned at the N-terminus exhibit multiple stability issues when compared to constructs wherein the scFv portion is positioned at the C-terminus.

Electrostatic surface of the bispecific antibody was calculated and charged patches were identified and disrupted. Extensive protein stability studies were performed and the constructed bispecific antibodies were screened for thermostability properties as well as CGRP and IL-23 binding (relative to the respective parental antibody) properties.

Chemical and physical modifications were therefore made to improve chemical and physical stability of the bispecific antibody of the present invention. Modifications to the parental IL-23 antibody, in scFv format, were made in HCDR4, HCDR5, LCDR4, LCDR5 and LCDR6 to improve chemical and physical stability. Constructed HCVR and LCVR were combined into the IL-23 scFv format according to the following formula: CGRP IgG (C-term.)-L1-HCVR2-L2-LCVR2. A disulfide bond, for stabilizing the IL-23 scFv, was engineered between the HCVR2 (G503C) and the LCVR2 (G694C) of the IL-23 scFv (numbering of amino acids applies linear numbering based on exemplified bispecific antibody presented in Tables 1(a) and (b)). Additionally, the parental CGRP antibody, in an IgG portion of the bispecific antibody, was engineered to an IgG₄ subclass because of a reduced ability to engage Fc receptor-mediated inflammatory mechanisms or to activate complement resulting in reduced effector function. The engineered IL-23 scFv construct and CGRP IgG construct, comprising these chemical and physical modifications, were inserted into an expression vector.

More specifically, the bispecific antibody of the present invention contains an IgG₄-PAA Fc portion. The IgG₄-PAA Fc portion has a serine to proline mutation at position 227 (S227P; SEQ ID NO: 1 or SEQ ID NO: 2), a phenylalanine to alanine mutation at position 233 (F233A; SEQ ID NO: 1 or SEQ ID NO: 2) and a leucine to alanine mutation at position 234 (L234A; SEQ ID NO: 1). The S227P mutation is a hinge mutation that prevents half-antibody formation (phenomenon of dynamic exchange of half-molecules in IgG₄ antibodies). The F233A and L234A mutations further reduce effector function of the already low human IgG₄ isotype.

A bispecific antibody containing a CGRP IgG, as an IgG₄ subclass, and an IL-23 scFv with six CDR mutations (relative to the parental IL-23 antibody described in U.S. Pat. No. 9,023,358: HCVR2 at K28P and T58V (SEQ ID NO: 6); and LCVR2 at L30G, L54K, E55L and M90Q/M90T (SEQ ID NO: 8 or SEQ ID NO: 9)(as represented in the exemplified bispecific antibody reflected in Tables 1(a) and (b): HCVR2 at K487P and T517V; and LCVR2 at L624G, L648K, E649L and M684Q/M684T, numbering of amino acids applies linear numbering based on exemplified bispecific antibody presented in Tables 1(a) and (b)) was identified as improving the expression, affinity (for IL-23 relative to the parental molecule) and thermostability issues demonstrated in initial constructs. The M90T mutation (SEQ ID NO: 9; (relative to the parental IL-23 antibody described in U.S. Pat. No. 9,023,358) has been found to improve the photostability of the molecule without adversely affecting binding to CGRP and IL-23p19. Additionally, these mutations resulted in a significantly reduced clearance rate in cynolomolgus monkeys. None of the above modifications were identified in initial characterizations of the parental single antibodies.

### Bispecific Antibody Binding and Activity

The bispecific antibodies of the present invention bind both human CGRP and human IL23p19 and neutralize at least one human CGRP bioactivity and at least one human IL-23 bioactivity in vitro or in vivo. The bispecific antibodies of the present invention are inhibitors of IL-23 in the presence and absence of CGRP in vitro. The bispecific antibodies of the present invention are inhibitors of CGRP in the presence or absence of IL-23 in vitro.

The first exemplified bispecific antibody of the present invention (Bispecific Antibody 1) is characterized as having a binding affinity (K_{D}) for human CGRP in the range of 26.0±26.0 pM and human IL23p19 in the range of 213.0±184.0 pM at 37° C.

The second exemplified bispecific antibody of the present invention (Bispecific Antibody 2) is characterized as having a binding affinity (K_{D}) for human CGRP of approximately 77.0 pM and human IL23p19 of 215 pM at 37° C.

The bispecific antibodies effectively neutralize CGRP and this neutralization is not affected by the presence of saturating amounts of human IL-23. The bispecific antibodies effectively neutralize human IL-23 and this neutralization is not affected by the presence of saturating amounts of human CGRP.

### Bispecific Antibody Expression

Expression vectors capable of direct expression of genes to which they are operably linked are well known in the art. Expression vectors can encode a signal peptide that facilitates secretion of the polypeptide(s) from a host cell. The signal peptide can be an immunoglobulin signal peptide or a heterologous signal peptide. The first polypeptide chain (comprising a HC, scFv, L1 and L2) and the second polypeptide chain (comprising a LC) may be expressed independently from different promoters to which they are operably linked in one vector or, alternatively, the first and second polypeptide chains may be expressed independently from different promoters to which they are operably linked in two vectors—one expressing the first polypeptide chain and one expressing the second polypeptide chain.

A host cell includes cells stably or transiently transfected, transformed, transduced or infected with one or more expression vectors expressing a first polypeptide chain, a second polypeptide chain or both a first and a second polypeptide chain of the invention. Creation and isolation of host cell lines producing a bispecific antibody of the invention can be accomplished using standard techniques known in the art. Mammalian cells are preferred host cells for expression of bispecific antibodies. Particular mammalian cells are HEK 293, NS0, DG-44, and CHO. Preferably, the bispecific antibodies are secreted into the medium in which the host cells are cultured, from which the bispecific antibodies can be recovered or purified.

It is well known in the art that mammalian expression of antibodies results in glycosylation. Typically, glycosylation occurs in the Fc region of the antibody at a highly conserved N-glycosylation site. N-glycans typically attach to asparagine. By way of example, each HC of exemplified bispecific antibody presented in Tables 1(a) and (b) is glycosylated at asparagine residue 296 of SEQ ID NO: 1 or SEQ ID NO: 2.

Medium, into which a bispecific antibody has been secreted, may be purified by conventional techniques. For example, the medium may be applied to and eluted from a Protein A or G column using conventional methods. Soluble aggregate and multimers may be effectively removed by common techniques, including size exclusion, hydrophobic interaction, ion exchange, or hydroxyapatite chromatography. The product may be immediately frozen, for example at −70° C., refrigerated, or may be lyophilized.

In some instances, a process for producing a bispecific antibody of the present invention may result in the formation of diabodies. Diabodies are bivalent formations of scFv in which HCVR2 and LCVR2 regions are expressed on a single polypeptide chain, but instead of the variable domains pairing with complementary domains of the same polypeptide chain, the variable domains pair with complementary domains of the other polypeptide chain or a different molecule. For example, if the bispecific antibody comprises two first polypeptides (for convenience, 1A and 1B, where each of 1A and 1B comprise a HC, a scFv, L1 and L2), and two second polypeptides (for convenience, 2A and 2B, where each of 2A and 2B comprise a LC), HCVR2 of 1A pairs with complementary domains of LCVR2 of 1B instead of pairing with LCVR2 of 1A.

### Therapeutic Uses

As used herein, “treatment” and/or “treating” are intended to refer to all processes wherein there may be a slowing, interrupting, arresting, controlling, or stopping of the progression of the disorders described herein, but does not necessarily indicate a total elimination of all disorder symptoms. Treatment includes administration of a bispecific antibody of the present invention for treatment of a disease or condition in a mammal, particularly in a human, that would benefit from a decreased level of CGRP and/or IL-23 or decreased bioactivity of CGRP and/or IL-23, and includes: (a) inhibiting further progression of the disease, i.e., arresting its development; and (b) relieving the disease, i.e., causing regression of the disease or disorder or alleviating symptoms or complications thereof.

The bispecific antibody of the present invention is expected to treat autoimmune diseases, including IBD (such as CD and UC), PsA and ankylosing spondylitis.

### Pharmaceutical Composition

A bispecific antibody of the invention can be incorporated into a pharmaceutical composition suitable for administration to a patient. A bispecific antibody of the invention may be administered to a patient alone or with a pharmaceutically acceptable carrier and/or diluent in single or multiple doses. Such pharmaceutical compositions are designed to be appropriate for the selected mode of administration, and pharmaceutically acceptable diluents, carrier, and/or excipients such as dispersing agents, buffers, surfactants, preservatives, solubilizing agents, isotonicity agents, stabilizing agents and the like are used as appropriate. Said compositions can be designed in accordance with conventional techniques disclosed in, e.g., Remington, The Science and Practice of Pharmacy, 22^{nd} Edition, Loyd V, Ed., Pharmaceutical Press, 2012, which provides a compendium of formulation techniques as are generally known to practitioners. Suitable carriers for pharmaceutical compositions include any material which, when combined with a bispecific antibody of the invention, retains the molecule's activity and is non-reactive with the patient's immune system. A pharmaceutical composition of the present invention comprises a bispecific antibody and one or more pharmaceutically acceptable carriers, diluents or excipients.

A pharmaceutical composition comprising a bispecific antibody of the present invention can be administered to a patient at risk for or exhibiting diseases or disorders as described herein using standard administration techniques.

A pharmaceutical composition of the invention contains an “effective” amount of a bispecific antibody of the invention. An effective amount refers to an amount necessary (at dosages and for periods of time and for the means of administration) to achieve the desired therapeutic result. An effective amount of the bispecific antibody may vary according to factors such as the disease state, age, sex, and weight of the individual, and the ability of the antibody or antibody portion to elicit a desired response in the individual. An effective amount is also one in which any toxic or detrimental effect of the bispecific antibody, are outweighed by the therapeutically beneficial effects.

### EXAMPLES

Except as noted otherwise, the exemplified bispecific antibody referred to throughout the Examples refers to the exemplified bispecific antibodies of the present invention set forth in Tables 1(a) and (b) above.

Bispecific Antibody Expression and Purification

An exemplified bispecific antibody of the present invention set forth in Tables 1(a) and (b) above (Bispecific Antibody 1) is expressed and purified essentially as follows. A glutamine synthetase (GS) expression vector containing a DNA polynucleotide sequence encoding for a polypeptide comprising the IgG HC-linker L1-scFv HCVR2-linker L2-scFv LCVR2 (polypeptide of SEQ ID NO: 1) and a second DNA polynucleotide sequence encoding a polypeptide comprising the IgG LC (polypeptide of SEQ ID NO: 3) is transfected into a GS-knockout Chinese hamster cell line (CHO) by electroporation. The expression vector encodes an SV Early promoter (Simian Virus 40E) and the gene for GS. Expression of GS allows for the biochemical synthesis of glutamine, an amino acid required by the CHO cells. Post-transfection cells undergo bulk selection with 50 μM L-methionine sulfoximine (MSX). The inhibition of GS by MSX is utilized to increase the stringency of selection. Cells with integration of the expression vector cDNA into transcriptionally active regions of the host cell genome can be selected against CHO wild type cells, which express an endogenous level of GS. Transfected pools are plated at low density to allow for close-to-clonal outgrowth of stable expressing cells. These masterwells are screened for bispecific antibody expression and then scaled up in serum-free suspension cultures to be used for production. Clarified medium, into which the exemplified bispecific antibody has been secreted, is applied to a Protein A affinity column that has been equilibrated with a compatible buffer such as 20 mM TRIS (pH 8.0). The column is washed to remove nonspecific binding components. The bound bispecific antibody is eluted, for example, by pH step or gradient such as 20 mM citrate (pH 3.0) and neutralized with Tris (pH 8) buffer. Bispecific antibody fractions are detected, such as by SDS-PAGE or analytical size-exclusion, and then are pooled. Soluble aggregate and multimers may be effectively removed by common techniques including size exclusion, hydrophobic interaction, ion exchange, or hydroxyapatite chromatography. Cation-exchange chromatography is used for Bispecific Antibody 1. Bispecific Antibody 1 is concentrated and/or sterile filtered using common techniques. The purity of Bispecific Antibody 1 after these chromatography steps is greater than 97% (monomer). The bispecific antibody may be immediately frozen at −70° C. or stored at 4° C. for several months.

The second exemplified bispecific antibody of the present invention (hereinafter referred to as Bispecific Antibody 2), which, relative to Bispecific Antibody 1, incorporates an engineered single amino acid change that substitutes threonine (T) for glutamine (Q) at position 684 (Q684T)(SEQ ID NO: 1 vs. SEQ ID NO: 2), is expressed in transiently transfected CHO and by Protein A and hydrophobic interaction chromatography. A glutamine synthetase (GS) expression vector containing a DNA polynucleotide sequence encoding for a polypeptide comprising the IgG HC-linker L1-scFv HCVR2-linker L2-scFv LCVR2 (polypeptide of SEQ ID NO: 2) and a second DNA polynucleotide sequence encoding a polypeptide comprising the IgG LC (polypeptide of SEQ ID NO: 3) is transiently transfected into GS-knockout Chinese hamster cell line (CHO) by chemical treatment with polyethyleimine. The remaining expression and purification steps are the same as for Bispecific Antibody 1. The purity of Bispecific Antibody 2 after these chromatography steps is greater than 98% (monomer).

Bispecific Antibody Binding Affinity to IL-23 and CGRP

Binding affinity of human CGRP and human IL-23 is determined by surface plasmon resonance (SPR) assay on a Biacore 3000 instrument primed with HBS-EP+(10 mM Hepes pH7.4+150 mM NaCl+3 mM EDTA+0.05% (w/v) surfactant P20) running buffer temperature controlled at 37° C. A CM5 chip (Biacore P/N BR-1000-12) containing immobilized protein A (generated using standard NHS-EDC amine coupling) on all four flow cells (Fc) was used to employ a capture methodology. Bispecific antibody samples are prepared at approximately 2 μg/mL by dilution into running buffer. Human IL-23 is prepared at final concentrations of 25, 12.5, 6.25, 3.13, 0.78, 0.39, 0.20, 0.10 and 0 (blank) nM by dilution into running buffer. Human CGRP is prepared at final concentrations of 12.5, 6.25, 3.13, 0.78, 0.39, 0.20, 0.10 and 0 (blank) nM by dilution into running buffer.

Each analysis cycle consists of (1) capturing different bispecific antibody samples on separate flow cells (Fc2, Fc3, and Fc4) at a level to facilitate a 20-100RU maximum response signal from either the human IL-23 or CGRP; (2) injection of each human IL-23 or human CGRP concentration over all four Fc at 100 μL/min for 120 seconds followed by return to buffer flow for 600 seconds to monitor dissociation phase; (3) regeneration of chip surfaces with injection of 10 mM glycine, pH 1.5, for 30 seconds at 10 μL/min over all cells; and (5) equilibration of chip surfaces in HBS-EP+ running buffer. Data are processed using standard double-referencing and fit to a 1:1 binding model using BiaEvaluation software, version 4.1, to determine the association rate (kₒₙ, M⁻¹s⁻¹ units), dissociation rate (k_{off}, s⁻¹ units), and Rₘₐₓ (RU units). The equilibrium dissociation constant (K_{D}) is calculated from the relationship K_{D}=k_{off}/kₒₙ, and is in molar units.

TABLE 2
Binding affinity of exemplified bispecific
antibodies to human IL-23 at 37° C.

| Molecule ID | kₒₙ (M⁻¹s⁻¹) | k_{off} (s⁻¹) | K_{D} (pM) |
|---|---|---|---|
| Bispecific Antibody 1 | 3.7 ± 3.8 × 10⁶ | 1.7 ± 1.1 × 10⁻⁴ | 210 ± 180* |
| (n = 4, average ± | | | |
| standard deviation) | | | |
| Bispecific Antibody 2 | 7.3 × 10⁵ | 1.6 × 10⁻⁴ | 220 |

*K_{D} calculated based on average of individual K_{D} values

TABLE 3
Binding affinity of exemplified bispecific
antibodies to human CGRP at 37° C.

| Molecule ID | kₒₙ (M⁻¹s⁻¹) | k_{off} (s⁻¹) | K_{D} (pM) |
|---|---|---|---|
| Bispecific Antibody 1 | 3.0 ± 3.2 × 10⁷ | 2.5 ± 1.3 × 10⁻⁴ | 26 ± 26** |
| (n = 2, average ± | | | |
| standard deviation) | | | |
| Bispecific Antibody 2 | 2.3 × 10⁶ | 1.8 × 10⁻⁴ | 77 |

**K_{D} calculated based on average of individual K_{D} values

These results demonstrate that the exemplified bispecific antibodies of the present invention bind human IL-23 and human CGRP at 37° C.

Bispecific Antibody Solubility and Stability Analysis

(a) Solubility

Bispecific Antibody 1 is dialyzed into 10 mM Citrate, pH 6 with and without 150 mM NaCl (abbreviated C6 and C6N respectively). Samples are concentrated to either 50 or approximately 100 mg/mL by centrifugation through a molecular weight filter (Amicon 30 kDa ultrafiltration filter, Millipore catalog # UFC903024). To a portion of both samples, Tween-80 is added to a final concentration of 0.02% (v/v; further abbreviated C6T and C6NT respectively). Select formulations are analyzed for solubility, freeze-thaw stability, and storage stability under refrigerated and room temperature conditions.

Solubility is characterized as bispecific antibody concentration >95 mg/mL in C6 and C6N formulations. After concentrating as described above, the samples are visually inspected at room temperature for precipitation or phase separation and subsequently stored for one week at 4° C. in the dark and visually re-inspected. This procedure is repeated on the same samples after storing for one additional week at −5° C. and for an additional week at −10° C. (note due to the level of dissolved substances the samples do not freeze). The results of the solubility analysis are shown in Table 4. Bispecific Antibody 1 showed no visual precipitation or phase separation in either formulation or storage temperatures.

TABLE 4
Solubility

| | | | After | | |
|---|---|---|---|---|---|
| Molecule | | Initial | 1 wk | After 1 wk | After 1 wk |
| ID | Formulation | (~25° C.) | at 4° C. | at −5° C. | at −10° C. |
| Bispecific | 108 mg/mL in | Clear | Clear | Clear | Clear |
| Antibody | C6 | | | | |
| 1 | 174 mg/mL in | Clear | Clear | Clear | Clear |
| | C6N | | | | |

(b) Freeze-Thaw Stability

During manufacturing the purified Active Pharmaceutical Ingredient (API) is typically held in a frozen state until forward processing into the Drug Product (DP). Bispecific Antibody 1 is tested for freeze-thaw stability at high concentration. A 50 and 100 mg/mL formulation in C6 and C6N are subjected to three slow freeze thaw cycles. The rate of freezing and thawing is controlled to mimic what would occur in a larger manufacturing container. A shelf lyophilizer under no vacuum is used to control the temperature cycle as shown in Table 5.

TABLE 5
Freeze and thaw rates used in slow freeze-thaw study

| | Target Temperature | Temperature change | Hold time at |
|---|---|---|---|
| Step | (° C.) | rate (° C./min) | Temperature (min) |
| 1 | 5 | 1.0 | 10 |
| 2 | −1 | 0.05 | 750 |
| 3 | −30 | 0.2 | 1 |
| 4 | −70 | 1.0 | 60 |
| 5 | −30 | 1.0 | 1 |
| 6 | −1 | 0.2 | 1000 |
| 7 | 0.5 | 0.2 | 1 |
| 8 | 15 | 1 | 1 |

After three cycles the material is analyzed by size exclusion chromatography (SEC) for high molecular weight (HMW) polymer formation and light obscuration for particles greater than 10 micron. Results are shown in Table 6. Bispecific Antibody 1 consistently yielded a low percentage of HMW polymer under all conditions tested.

TABLE 6
Stability against three slow freeze-thaw cycles (nd = not determined)

| | | % HMW | Particle Count/mL |
|---|---|---|---|
| Molecule ID | Formulation | increase | (≥10 micron) |
| Bispecific | 50 mg/mL in C6T | 1.8 | 573 |
| Antibody 1 | 50 mg/mL in C6NT | 1.0 | 536 |
| | 100 mg/mL in C6T | 2.2 | nd |
| | 100 mg/mL in C6NT | 1.4 | nd |

(c) Refrigerated and Room Temperature Stability

Refrigerated and room temperature stability under a generic Drug Product (DP) formulation, 10 mM citrate, 0.02% Tween-80, pH 6.0 with and without 150 mM NaCl (abbreviated C6T and C6NT respectively) is evaluated by SEC and particle counts following two and four week hold time. Results are shown in Tables 7 and 8, respectively. Data demonstrate that Bispecific Antibody 1 has low soluble (% HMW) and insoluble (>10 micron particle count) stability.

TABLE 7
Stability in generic drug product (DP) formulation at 50 mg/mL, HMW
formation

| | | % HMW increase | % HMW increase |
|---|---|---|---|
| | Formulation, | follow 4° C. | follow 25° C. |
| Molecule ID | Incubation | storage | storage |
| Bispecific | 2 wk in C6T | −0.1 | 0.5 |
| Antibody 1 | 4 wk in C6T | 0.0 | 0.7 |
| | 2 wk in C6NT | −0.2 | −0.1 |
| | 4 wk in C6NT | −0.2 | 0.0 |

TABLE 8
Stability in generic drug product (DP) formulation
at 50 mg/mL, micron size particle formation

| | Formulation, | ≥10 micron particles/mL |
|---|---|---|
| Molecule ID | Incubation | follow 25° C. storage |
| Bispecific Antibody 1 | 2 wk in C6T | 67 |
| | 4 wk in C6T | 373 |
| | 2 wk in C6NT | 98 |
| | 4 wk in C6NT | 129 |

(d) Viscosity

Viscosity of Bispecific Antibody 1 is analyzed at 100 mg/mL in four formulations (C6, C6N, C6T, and C6NT) at room temperature. Measurements are made on a m-VROC (Rheosense) using a shear rate of 1000 sec-1 at 25° C. Results are shown in Table 9 and illustrate significantly low viscosity for Bispecific Antibody 1 in both C6N and C6NT formulations. Significant reduction in viscosity is observed for Bispecific Antibody 1 in salt-containing formulations.

TABLE 9
Solution viscosity of 100 mg/mL Bispecific Antibody
1 at room temperature in various formulations

| Molecule ID | C6 | C6N | C6T | C6NT |
|---|---|---|---|---|
| Bispecific Antibody 1 | 9.2 | 2.9 | 13.2 | 3.8 |

(e) Photostability

Photostability of the Bispecific Antibody 1 and Bispecific Antibody 2 are characterized at 50 mg/mL protein concentration under one formulation condition (C6NT). Bispecific Antibody 1 is exposed to 20% of the International Conference on Harmonization (ICH) Expert Working Group recommend exposure level (Q1B-Stability Testing: Photostability Testing of New Drug Substances and Products, November 1996). This equates to 240,000 lux-hours of visible light and 40 watt-hour/m² near-UV light. A Bahnson ES2000 photochamber (Environmental Specialties, a Bahnson Group Company) equipped with catalog 04030-307-CW visible and 04030-308UV near-UV lamps is used. Samples are exposed to visible light at 8,000 lux intensity for 30 hours and 10 watt/m² near-UV light for 4 hours. All exposures are at 25° C. in type I borosilicate glass HPLC vials. Following exposure, the percent HMW polymer formation is determined by SEC and is shown in Table 10. The results demonstrate that the Q684T mutation (SEQ ID NO: 1 vs. SEQ ID NO: 2) in Bispecific Antibody 2 significantly improves the photostability of the molecule.

TABLE 10
Photostability of Bispecific Antibody 1 at 50 mg/mL in C6NT formulation

| | % HMW increase | % HMW increase (240,000 lux-hr |
|---|---|---|
| | (240,000 lux-hr | visible plus 40 watt-hr/m² near- |
| Molecule ID | visible) | UV) |
| Bispecific | 5.5% | 11.7 ± 2.2 |
| Antibody 1 | | (n = 3, average ± standard deviation) |
| Bispecific | not determined | 5.3 |
| Antibody 2 | | |

Simultaneous Binding of IL-23 and CRGP

A BIAcore 3000 instrument (GE Healthcare Life Sciences) is used to determine if the bispecific antibodies of the present invention can bind to human IL-23 and human CGRP simultaneously. The instrument is primed with HBS-EP+(10 mM Hepes pH 7.4+150 mM NaCl+3 mM EDTA+0.05% (w/v) surfactant P20) running buffer equilibrated at 25° C. A CM5 chip (Biacore P/N BR1000-12) containing immobilized Protein A (generated using standard NHS-EDC amine coupling) on all four flow cells (Fc) is used to employ a bispecific antibody capture methodology. Bispecific Antibodies 1 and 2 are diluted in running buffer and injected over individual flow lanes to capture approximately 900RU of antibody. Human CGRP at 10 nM in running buffer is injected over the bispecific antibody surfaces and binding observed. To ensure that all CGRP binding sites in the bispecific antibodies are saturated, additional injections of 20 and then 40 nM CGRP peptide are made. No to minimal increase in binding signal is observed certifying that all available anti-CGRP binding sites are occupied. Thereafter, a 150 nM solution of human IL-23 is injected. If the bispecific antibody is capable of simultaneously binding both CGRP and IL-23, a signal increase should be observed. For Bispecific Antibodies 1 and 2 a significant increase in binding signal is observed thus demonstrating that these bispecific antibodies are capable of simultaneously binding both human IL-23 and human CGRP.

Bispecific Antibody 1 does not Bind to Human IL-12

A BIAcore 2000 instrument is used to determine if the bispecific antibody of the invention will bind human IL-12. Unless noted, reagents and materials are purchased from GE Healthcare Life Sciences (Upsala, Sweden); measurements performed at 25° C., and HBS-P buffer (150 mM sodium chloride, 0.005% (w/v) surfactant P-20, and 10 mM HEPES, pH 7.4) is used as the running- and sample-buffer. Protein A (Calbiochem) is immobilized on flow cells 1, 2, 3 and 4 of a CM5 sensor chip using an amine coupling kit. Bispecific Antibody 1 (diluted to 2 μg/mL) is captured first on flow cell 2 (with a 5 second injection at 80 μL/min, yielding 460 response units (Δ RU) of Bispecific Antibody 1 capture). Flow cell 1 is a protein-A-only control. Next, human IL-12 (Peprotech) is injected (667 nM) for 2 minutes and no additional binding signal (0 Δ RU) is observed. A commercial antibody specific for IL-12 (anti-human IL-12 antibody sold under the trade name STELARA®) binds human IL-12.

The results demonstrate that Bispecific Antibody 1 does not bind human IL-12. Moreover, using the same chip, the anti-IL-12 specific antibody binds to human IL-12.

Inhibition of IL-23-Mediated Stat 3 Activity In Vitro in Kit225 Cells

Kit225 is a human T-cell line established from a patient with T-cell chronic lymphocytic leukemia. Kit225 cells naturally express IL-23R and respond to human IL-23 by phosphorylation of STAT3 and activation of the STAT3 pathway. The ability of IL-23 to activate STAT3 pathway is assessed by measuring luciferase activity in Kit225 cells stably transfected with STAT3-luciferase construct.

Kit225-Stat3-luc (clone 3) cells are routinely cultured in assay medium (RPMI 1640 containing 10% FBS, 10 ng/mL human IL-2, and 1× penicillin plus puromycin). On the day of assay, the cells are collected by centrifugation at 500×g for 5 minutes (RT), washed with large volume of serum free RPMI 1640 medium and re-suspended in serum free OPTI-MEM medium. 50,000 Kit225 cells per well (in 50 μL) are added to the wells of a white/clear bottom TC treated 96 well plate and treated with the antibodies in the presence of human IL-23.

For each test 25 μL of a 4× antibody solution are added per well. A dose range of Bispecific Antibody 1 from 0 to 126790 pM is evaluated (final concentration based on the MW of Bispecific Antibody 1; MW=197178 Da). 25 μL of 4× human IL-23 (hIL-23) is added to each well to a final concentration of 50 pM (based on MW=60000 Da). The assay medium alone is used for “medium alone” and “hIL-23 alone” control. An IL-23 neutralizing antibody (Positive Control Antibody), targeting the p19 subunit of IL-23, tested in a dose range from 0 to 100000 pM (final concentration base on MW of Antibody 2 MW=150000 Da) is used as a positive control. The Isotype Control Antibody (human IgG4-PAA) tested in a dose range from 0 to 126, 790 pM (final concentration base on MW=150000 Da) is used as a negative control. Testing is carried in triplicate. 96-well plates are placed in tissue culture incubator (37° C., 95% relative humidity, 5% CO2) for 4 hours. 100 μL/well of Bright-Glo Luciferase solution (Promega) is added to stop the assay upon the treatment Luminometer (Perkin Elmer Victor3) is used to read the plates. Results are expressed as the concentration where 50% of the IL-23-induced Stat 3 activity is inhibited (IC₅₀) by either Bispecific Antibody 1 or the Positive Control Antibody and is calculated using a 4 parameter sigmoidal fit of the data (Sigma plot).

The results demonstrate that Antibody 1 inhibits human IL-23 induced Stat 3 activity in Kit225 cells in a concentration-dependent manner. Inhibition is comparable to that observed with the Positive Control Antibody (with an IC₅₀ for Bispecific Antibody 1 of 1671±236 pM versus 466±31 pM for the Positive Control anti-IL-23p19 antibody.

Addition of 50 nM of CGRP to the assay does not modify the activity of the Bispecific Antibody 1, as the IC₅₀ in presence of CGRP is comparable to that described above.

Negative control antibody does not inhibit Stat 3 activity in Kit225 cells at any concentration tested.

Bispecific Antibody 1 effectively neutralizes human IL-23 and IL-23 inhibition is not affected by presence of CGRP.

Inhibition of cAMP Production Induced by CGRP in SK-N-MC Cells In Vitro

SK-N-MC cells are a human neuroblastoma cell line that endogenously expresses the CGRP receptor. This receptor is functionally coupled to intracellular Gas proteins. Stimulation of the receptor with its natural agonist, CGRP peptide, results in an increased synthesis of cAMP. Because the amount of cAMP present within cells can be detected using standard in vitro technology, this parameter is used as a measure of receptor activity.

Cultured SK-N-MC are grown in MEM (Hyclone) supplemented with 10% heat-inactivated FBS (Gibco), Non-Essential Amino Acids (Gibco), 1 mM sodium pyruvate, 2 mM L-glutamine, 100 U/mL of penicillin, and 10 μg/mL of streptomycin to about 70% confluence. After providing fresh medium, the cells are incubated at 37° C. overnight. On the day of the assay, cells are detached using Accutase (MP Biomedicals), resuspended in assay buffer (HBSS/DPBS with Mg⁺⁺ and Ca⁺⁺ mixed 1:2, 3.3 mM HEPES, 0.03% BSA, 0.5 mM IBMX), and seeded 3-5K/well into 384-well, poly-D-lysine coated white plates (BD Biosciences).

Bispecific Antibody 1 is diluted 1:3 in assay buffer from 10 nM to 0.5 pM (MW of bispecific antibody is 200 kDa). Diluted Bispecific Antibody 1, Positive Control Antibody (a CGRP neutralizing antibody described in U.S. Pat. No. 9,073,991) or an Isotype Control Antibody (human IgG4-PAA) are mixed with Human IL-23 (10 nM, final concentration) or an equal volume of buffer and incubated with the cells for 30 minutes at room temperature. Human CGRP peptide (Bachem H-1470) is added at its EC₈₀ concentration (0.8 nM), and the plates are incubated for 60 minutes at room temperature. The signal window is established using 10 nM BIBN 4096 (Tocris), a small molecule reference antagonist (Kb=0.01 nM). The amount of intracellular cAMP is quantitated using HTRF technology (Homogeneous Time Resolved Fluorescence; Cisbio) as per vendor instructions. Briefly, cAMP-d2 conjugate and anti-cAMP-cryptate conjugate in lysis buffer are incubated with the treated cells at room temperature for 60-90 minutes. The HTRF signal is immediately detected using an EnVision plate reader (Perkin-Elmer) to calculate the ratio of fluorescence at 665 to 620 nM. The raw data are converted to cAMP amount (pmole/well) using a cAMP standard curve generated for each experiment. Relative EC₅₀ values are calculated from the top-bottom range of the concentration response curve using a four-parameter logistic curve fitting program (ActivityBase v5.3.1.22 or Genedata Screener v12.0.4), and Kb values are estimated as agonist-corrected IC50 values using the equation:

Kb=(IC50)/[1+([Ag]/EC50)].

The results demonstrate that Bispecific Antibody 1 inhibits CGRP-stimulated cAMP production in a dose-dependent manner, with an estimated Kb of 0.02 nM and a maximum effect equal to that produced by a reference antagonist and the Positive Control Antibody. The presence of 10 nM human IL-23 had no effect on the inhibition by Bispecific Antibody 1 or the Positive Control Antibody. The Isotype Control Antibody did not inhibit CGRP-induced cAMP production at any concentration tested.

TABLE 11
Inhibition of CGRP-induced cAMP production by test antibodies

| | |
|---|---|
| | Maximum |
| Kb (nM) | % Activity |

| Antibody | −IL23 | +IL-23 | −IL23 | +IL-23 |
|---|---|---|---|---|
| Antibody 1 | 0.02 | 0.02 | 99.8 | 99.9 |
| Isotype Control | >2.5 | >2.5 | 3.3 | 6.9 |
| Positive Control | 0.02 | 0.02 | 99.9 | 99.8 |

Inhibition of Human IL-23-Induced Mouse IL-22 Production In Vivo

Administration of human IL-23 induces expression of mouse IL-22 in normal Balb/c mice in vivo.

To understand if Bispecific Antibody 1 will block human IL-23-induced expression of mouse IL-22, in vivo, normal Balb/c mice (N=5) are injected intraperitoneally with either 67 nm/kg of exemplified Bispecific Antibody 1 (molecular weight 200 kDa) or with a Isotype Control Antibody (human IgG4-PAA used as negative control antibody, 67 nmol/kg, molecular weight 150 kDa). Two days post-injection, mice are challenged by intraperitoneal injection of 50 nmol/kg of human IL-23. Five hours post-human IL-23 challenge mice are sacrificed and serum is collected. Collected serum is analyzed for mouse IL-22 expression using commercial ELISA (eBioscience, Cat. #88-7422-88) according to manufacturer's instructions.

TABLE 12
Inhibition of human IL-23-induced mouse IL-22 production in vivo

| | Bispecific | | Isotype Control |
|---|---|---|---|
| | Antibody 1 + | | Antibody + human |
| | human IL-23 | Naïve mouse | IL-23 |
| mouse IL-22 Levels | 3.6 ± 2.2 | 0 ± 0 | 577 ± 57 |
| (pg/mL) | | | |

The results demonstrate that Bispecific Antibody 1 blocks the human IL-23-induced increase in mIL-22 expression. The mouse IL-22 levels in the serum of mice treated with the Bispecific Antibody 1 are comparable to mouse IL-22 levels observed in the serum of naïve mice (p<0.0001, t test with unequal variance). The Isotype Control Antibody does not inhibit human IL-23-induced expression of mouse IL-22. Bispecific Antibody 1 effectively neutralizes human IL-23 in vivo.

Administration of the Bispecific Antibody 1 Prevents Capsaicin-Induced Increase in Rat Dermal Blood Flow

The capsaicin induced Laser Doppler Imaging (LDI) blood flow method is based on a capsaicin solution topically applied to the skin that induces inflammation, which is detected by a local change in blood flow that can be monitored using LDI. This method is dependent on capsaicin activation of the Transient Receptor Potential cation channel subfamily V member 1 (TRPV1) receptor followed by a local release of CGRP and activation of the CGRP receptor on the blood vessels in the skin. The capsaicin-induced dermal vasodilation model has been applied to assess target engagement in pre-clinical (rat, non-human primate (NHP)) models and is translational to the clinic. The purpose of this study is to determine if Bispecific Antibody 1 is able to prevent CGRP-mediated capsaicin-induced dermal vasodilation in the rat abdominal skin.

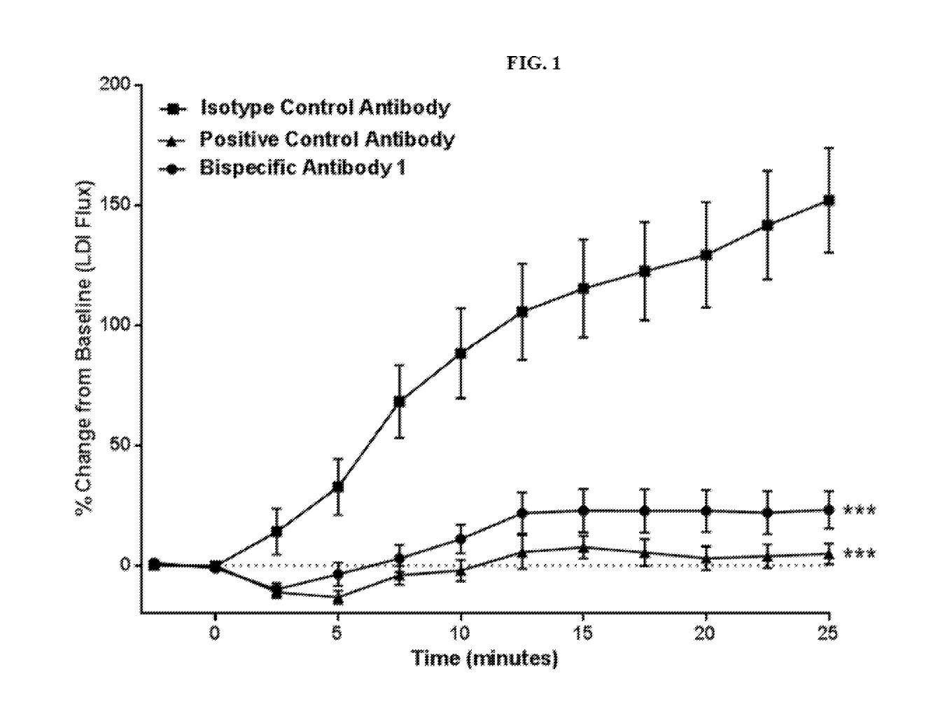

Bispecific Antibody 1, a Positive Control (a CGRP neutralizing antibody described in U.S. Pat. No. 9,073,991) and Isotype Control Antibody (human IgG4-PAA) are prepared in PBS. Lewis Rats are treated (n=8 per group) with the respective antibodies subcutaneously at 4 mg/kg 5 days prior to the LDI measurement and fasted overnight prior to the experiment. Study operators are blinded to the treatments. On the day of the experiment, the rat abdomens are shaved and the rats placed in a heated air chamber on a heating pad under the LDI instrument (Moor LDI Laser Doppler Imager, Model LDI2-IR). A rectal thermometer and blood pressure cuffs are used throughout the study for temperature and BP monitoring. The rats are stabilized under 2.0±0.5% Isoflurane anesthesia for approximately 20 minutes prior to scanning. During this stabilization period, preliminary scans are obtained for correct positioning of three neoprene O-rings (away from visible blood vessels and high basal blood flow areas). Once baseline temperature (approximately 37° C.) is stabilized, imaging scans begin with 2 baseline scans. After the second scan is completed, 8 μL of capsaicin solution is applied to each of the three O-rings (50 mg of capsaicin in a solution of 600 μL EtOH, 40 μL Tween 20 and 100 μL purified H₂O). Scanning continues with a scan every 2.5 minutes for an additional 25 minutes. Once scans are complete, a blood sample is obtained via cardiac puncture for plasma analysis. LDI repeat scans are analyzed using Moor v.5.3 software for region of interest analysis and Microsoft Excel worksheets for averaging the signal from the region of interests at a given time point and analysis of changes in blood flow reported as percent change from baseline (baseline value was the average of two baseline scans). Analyzed data is entered into Graphpad Prism 6 for graphing. ANOVA followed by Tukey's multiple comparisons is used for statistical analysis. The results are illustrated in FIG. 1 (n=8, ANOVA with Tukey's multiple comparisons, ***p<0.001 compared with Isotype Control Antibody).

Bispecific Antibody 1 and the Positive Control Antibody inhibit CGRP-mediated capsaicin-induced dermal vasodilation. In contrast, the Isotype Control Antibody does not inhibit CGRP-mediated capsaicin-induced dermal vasodilation.

Nonclinical PK of Bispecific Antibody 1 in Monkey

Serum pharmacokinetics of Bispecific Antibody 1 is determined as follows: male cynomolgus monkeys are administered 6.7 mg/kg of Bispecific Antibody 1 (prepared in solution of PBS (PH7.4)) of the invention either intravenously (N=1) or subcutaneously (N=2).

Blood samples (˜1 mL) are collected (intravenously from a femoral vein into serum separator tubes (e.g., containing no anticoagulant) and processed to serum) are collected pre-dosing and subsequently, post-dosing at 1-, 6-, 12-, 24-, 48-, 72-, 96-, 120-, 144-, 168-, 240-, 336-, 504-, and 672-hours. Serum samples are analyzed by quantitative MS for total IgG. Samples are immunoprecipitated with biotinylated goat anti-hIgG (Southern Biotech, 2049-08) and streptavidin coated magnetic beads. Following immunoprecipitation, samples are reduced, alkylated, and digested with trypsin. Total IgG concentrations are determined using selected tryptic peptides as a surrogate measure of antibody exposure. Detection and integration of data are performed using a Thermo Q-Exactive Orbitrap LC/MS system.

Standard curves for the tested antibody are generated by dilution of known amounts of exemplified bispecific antibody into 100% Cynomolgus monkey serum (Bioreclamation). A standard curve range of Bispecific Antibody 1 is 25-12,800 ng/mL (with an upper and lower limit of quantification of 12,800 ng/mL and 25 ng/mL, respectively).

Pharmacokinetic parameters (clearance values) are calculated using concentration versus time profile from time zero (administration of antibody) to 672 hours post-administration and are determined via non-compartmental analysis using Phoenix (WinNonLin 6.4, Connect 1.4). The results are summarized in Table 13.

TABLE 13
Clearance of Bispecific Antibody 1 in cynomolgus monkey
following single intravenous or subcutaneous administration

| Antibody | Intravenous Clearance | Subcutaneous Clearance |
|---|---|---|
| Administered | (mL/hr/kg) | (mL/hr/kg) |
| Bispecific Antibody 1 | 0.448 | 0.768 |

SEQUENCES

First Encoded Polypeptide; IgG HC, L1, scFv HCVR2, L2, and scFv LCVR2
(SEQ ID NO: 1)
QVQLVQSGAEVKKPGSSVKVSCKASGYTFGNYWMQWVRQAPGQGLEWMGAI
YEGTGKTVYIQKFADRVTITADKSTSTAYMELSSLRSEDTAVYYCARLSDYVSGF
GYWGQGTTVTVSSASTKGPSVFPLAPCSRSTSESTAALGCLVKDYFPEPVTVSWN
SGALTSGVHTFPAVLQSSGLYSLSSVVTVPSSSLGTKTYTCNVDHKPSNTKVDKR
VESKYGPPCPPCPAPEAAGGPSVFLFPPKPKDTLMISRTPEVTCVVVDVSQEDPEV
QFNWYVDGVEVHNAKTKPREEQFNSTYRVVSVLTVLHQDWLNGKEYKCKVSN
KGLPSSIEKTISKAKGQPREPQVYTLPPSQEEMTKNQVSLTCLVKGFYPSDIAVEW
ESNGQPENNYKTTPPVLDSDGSFFLYSRLTVDKSRWQEGNVFSCSVMHEALHNH
YTQKSLSLSLGGGGSGGGGSGGGGSQVQLVQSGAEVKKPGSSVKVSCKASGYPF
TRYVMHWVRQAPGQCLEWMGYINPYNDGVNYNEKFKGRVTITADESTSTAYM
ELSSLRSEDTAVYYCARNWDTGLWGQGTTVTVSSGGGGSGGGGSGGGGSGGGG
SDIQMTQSPSSLSASVGDRVTITCKASDHIGKFLTWYQQKPGKAPKLLIYGATSKL
TGVPSRFSGSGSGTDFTLTISSLQPEDFATYYCQQYWSTPFTFGCGTKVEIK
Second Encoded Polypeptide; IgG HC, L1, scFv HCVR2, L2, and scFv LCVR2
(SEQ ID NO: 2)
QVQLVQSGAEVKKPGSSVKVSCKASGYTFGNYWMQWVRQAPGQGLEWMGAI
YEGTGKTVYIQKFADRVTITADKSTSTAYMELSSLRSEDTAVYYCARLSDYVSGF
GYWGQGTTVTVSSASTKGPSVFPLAPCSRSTSESTAALGCLVKDYFPEPVTVSWN
SGALTSGVHTFPAVLQSSGLYSLSSVVTVPSSSLGTKTYTCNVDHKPSNTKVDKR
VESKYGPPCPPCPAPEAAGGPSVFLFPPKPKDTLMISRTPEVTCVVVDVSQEDPEV
QFNWYVDGVEVHNAKTKPREEQFNSTYRVVSVLTVLHQDWLNGKEYKCKVSN
KGLPSSIEKTISKAKGQPREPQVYTLPPSQEEMTKNQVSLTCLVKGFYPSDIAVEW
ESNGQPENNYKTTPPVLDSDGSFFLYSRLTVDKSRWQEGNVFSCSVMHEALHNH
YTQKSLSLSLGGGGSGGGGSGGGGSQVQLVQSGAEVKKPGSSVKVSCKASGYPF
TRYVMHWVRQAPGQCLEWMGYINPYNDGVNYNEKFKGRVTITADESTSTAYM
ELSSLRSEDTAVYYCARNWDTGLWGQGTTVTVSSGGGGSGGGGSGGGGSGGGG
SDIQMTQSPSSLSASVGDRVTITCKASDHIGKFLTWYQQKPGKAPKLLIYGATSKL
TGVPSRFSGSGSGTDFTLTISSLQPEDFATYYCQTYWSTPFTFGCGTKVEIK
Third Encoded Polypeptide; IgG LC (SEQ ID NO: 3)
DIQMTQSPSSLSASVGDRVTITCRASKDISKYLNWYQQKPGKAPKLLIYYTSGYH
SGVPSRFSGSGSGTDFTLTISSLQPEDFATYYCQQGDALPPTFGGGTKVEIKRTVA
APSVFIFPPSDEQLKSGTASVVCLLNNFYPREAKVQWKVDNALQSGNSQESVTEQ
DSKDSTYSLSSTLTLSKADYEKHKVYACEVTHQGLSSPVTKSFNRGEC
IgG Heavy Chain (SEQ ID NO: 4)
QVQLVQSGAEVKKPGSSVKVSCKASGYTFGNYWMQWVRQAPGQGLEWMGAI
YEGTGKTVYIQKFADRVTITADKSTSTAYMELSSLRSEDTAVYYCARLSDYVSGF
GYWGQGTTVTVSSASTKGPSVFPLAPCSRSTSESTAALGCLVKDYFPEPVTVSWN
SGALTSGVHTFPAVLQSSGLYSLSSVVTVPSSSLGTKTYTCNVDHKPSNTKVDKR
VESKYGPPCPPCPAPEAAGGPSVFLFPPKPKDTLMISRTPEVTCVVVDVSQEDPEV
QFNWYVDGVEVHNAKTKPREEQFNSTYRVVSVLTVLHQDWLNGKEYKCKVSN
KGLPSSIEKTISKAKGQPREPQVYTLPPSQEEMTKNQVSLTCLVKGFYPSDIAVEW
ESNGQPENNYKTTPPVLDSDGSFFLYSRLTVDKSRWQEGNVFSCSVMHEALHNH
YTQKSLSLSL
IgG Heavy Chain Variable Region 1 (HCVR1)(SEQ ID NO: 5)
QVQLVQSGAEVKKPGSSVKVSCKASGYTFGNYWMQWVRQAPGQGLEWMGAI
YEGTGKTVYIQKFADRVTITADKSTSTAYMELSSLRSEDTAVYYCARLSDYVSGF
GYWGQGTTVTVSS
scFv Heavy Chain Variable Region 2 (HCVR2)(SEQ ID NO: 6)
QVQLVQSGAEVKKPGSSVKVSCKASGYPFTRYVMHWVRQAPGQCLEWMGYIN
PYNDGVNYNEKFKGRVTITADESTSTAYMELSSLRSEDTAVYYCARNWDTGLW
GQGTTVTVSS
IgG Light Chain Variable Region 1 (LCVR1)(SEQ ID NO: 7)
DIQMTQSPSSLSASVGDRVTITCRASKDISKYLNWYQQKPGKAPKLLIYYTSGYH
SGVPSRFSGSGSGTDFTLTISSLQPEDFATYYCQQGDALPPTFGGGTKVEIK
scFv Light Chain Variable Region 2 (LCVR2)(SEQ ID NO: 8)
DIQMTQSPSSLSASVGDRVTITCKASDHIGKFLTWYQQKPGKAPKLLIYGATSKLT
GVPSRFSGSGSGTDFTLTISSLQPEDFATYYCQQYWSTPFTFGCGTKVEIK
scFv Light Chain Variable Region 2 (SEQ ID NO: 9)
DIQMTQSPSSLSASVGDRVTITCKASDHIGKFLTWYQQKPGKAPKLLIYGATSKLT
GVPSRFSGSGSGTDFTLTISSLQPEDFATYYCQTYWSTPFTFGCGTKVEIK
HCDR1 (SEQ ID NO: 10)
KASGYTFGNYWMQ
HCDR2 (SEQ ID NO: 11)
AIYEGTGKTVYIQKFAD
HCDR3 (SEQ ID NO: 12)
ARLSDYVSGFGY
HCDR4 (SEQ ID NO: 13)
KASGYPFTRYVMH
HCDR5 (SEQ ID NO: 14)
YINPYNDGVNYNEKFKG
HCDR6 (SEQ ID NO: 15)
ARNWDTGL
LCDR1 (SEQ ID NO: 16)
RASKDISKYLN
LCDR2 (SEQ ID NO: 17)
YYTSGYHS
LCDR3 (SEQ ID NO: 18)
QQGDALPPT
LCDR4 (SEQ ID NO: 19)
KASDHIGKFLT
LCDR5 (SEQ ID NO: 20)
YGATSKLT
LCDR6 (SEQ ID NO: 21)
QQYWSTPFT
LCDR6 (SEQ ID NO: 22)
QTYWSTPFT
Polypeptide Linker 1 (SEQ ID NO: 23)
GGGGSGGGGSGGGGS
Polypeptide Linker 2 (SEQ ID NO: 24)
GGGGSGGGGSGGGGSGGGGS
FRH1-1 (SE(Q ID NO: 25)
QVQLVQSGAEVKKPGSSVKVSC
FRH1-2 (SEQ ID NO: 26)
WVRQAPGQGLEWMG
FRH1-3 (SEQ ID NO: 27)
RVTITADKSTSTAYMELSSLRSEDTAVYYC
FRH1-4 (SEQ ID NO: 28)
WGQGTTVTVSS
FRH2-1 (SEQ ID NO: 29)
QVQLVQSGAEVKKPGSSVKVSC
FRH2-2 (SEQ ID NO: 30)
WVRQAPGQCLEWMG
FRH2-3 (SEQ ID NO: 31)
RVTITADESTSTAYMELSSLRSEDTAVYYC
FRH2-4 (SEQ ID NO: 32)
WGQGTTVTVSS
FRL2-1 (SEQ ID NO: 33)
DIQMTQSPSSLSASVGDRVTITC
FRL2-2 (SEQ ID NO: 34)
WYQQKPGKAPKLLI
FRL2-3 (SEQ ID NO: 35)
GVPSRFSGSGSGTDFTLTISSLQPEDFATYYC
FRL2-4 (SEQ ID NO: 36)
FGCGTKVEIK
FRL1-1 (SEQ ID NO: 37)
DIQMTQSPSSLSASVGDRVTITC
FRL1-2 (SEQ ID NO: 38)
WYQQKPGKAPKLLI
FRL1-3 (SEQ ID NO: 39)
GVPSRFSGSGSGTDFTLTISSLQPEDFATYYC
FRL1-4 (SEQ ID NO: 40)
FGGGTKVEIK
HEAVY CHAIN CONSTANT REGION (SEQ ID NO: 41)
ASTKGPSVFPLAPCSRSTSESTAALGCLVKDYFPEPVTVSWNSGALTSGVHTFPA
VLQSSGLYSLSSVVTVPSSSLGTKTYTCNVDHKPSNTKVDKRVESKYGPPCPPCP
APEAAGGPSVFLFPPKPKDTLMISRTPEVTCVVVDVSQEDPEVQFNWYVDGVEV
HNAKTKPREEQFNSTYRVVSVLTVLHQDWLNGKEYKCKVSNKGLPSSIEKTISK
AKGQPREPQVYTLPPSQEEMTKNQVSLTCLVKGFYPSDIAVEWESNGQPENNYK
TTPPVLDSDGSFFLYSRLTVDKSRWQEGNVFSCSVMHEALHNHYTQKSLSLSL
LIGHT CHAIN CONSTANT REGION (SEQ ID NO: 42)
RTVAAPSVFIFPPSDEQLKSGTASVVCLLNNFYPREAKVQWKVDNALQSGNSQES
VTEQDSKDSTYSLSSTLTLSKADYEKHKVYACEVTHQGLSSPVTKSFNRGEC

1. A bispecific antibody comprising an immunoglobulin G (IgG) antibody and two single chain variable fragments (scFv) wherein,
(a) said IgG comprises two heavy chains (HC) and two light chains (LC), each HC comprises a heavy chain variable region (HCVR1) comprising heavy chain CDRs (HCDR) 1-3 and each light chain comprises a light chain variable region (LCVR1) comprising light chain CDRs (LCDR) 1-3, wherein the amino acid sequence of HCDR1 is SEQ ID NO: 10, the amino acid sequence of HCDR2 is SEQ ID NO: 11, the amino acid sequence of HCDR3 is SEQ ID NO: 12, the amino acid sequence of LCDR1 is SEQ ID NO: 16, the amino acid sequence of LCDR2 is SEQ ID NO: 17, and the amino acid sequence of LCDR3 is SEQ ID NO: 18; and
(b) each scFv comprises a heavy chain variable region (HCVR2) and a light chain variable region (LCVR2), the HCVR2 comprising HCDRs 4-6, and the LCVR2 comprising LCDRs 4-6, wherein the amino acid sequence of HCDR4 is SEQ ID NO: 13, the amino acid sequence of HCDR5 is SEQ ID NO: 14, the amino acid sequence of HCDR6 is SEQ ID NO: 15, the amino acid sequence of LCDR4 is SEQ ID NO: 19, the amino acid sequence of LCDR5 is SEQ ID NO: 20, and the amino acid sequence of LCDR6 is SEQ ID NO: 21 or SEQ ID NO: 22,
wherein each scFv is linked at the N-terminus of HCVR2 of each scFv to said IgG antibody at the C-terminus of each IgG HC via a polypeptide linker (L1),
wherein the HCVR2 of each scFv is linked at the C-terminus of the HCVR2 to the LCVR2 of the same scFv at the N-terminus of the LCVR2 of the same scFv via a second polypeptide linker (L2)
and wherein the bispecific antibody binds to human calcitonin gene related peptide (CGRP) and the p19 subunit of human IL-23.

2. The bispecific antibody according to claim 1, wherein the amino acid sequence of LCDR6 is SEQ ID NO: 21.

3. The bispecific antibody according to claim 1, wherein the amino acid sequence of LCDR6 is SEQ ID NO: 22.

4. The bispecific antibody according to claim 1, wherein the amino acid sequence of HCVR1 of each HC is SEQ ID NO: 5, the amino acid sequence of LCVR1 of each LC is SEQ ID NO: 7, the amino acid sequence of HCVR2 of each scFv is SEQ ID NO: 6 and the amino acid sequence of LCVR2 of each scFv is SEQ ID NO: 8 or SEQ ID NO: 9.

5. The bispecific antibody according to claim 4, wherein the amino acid sequence of LCVR2 of each scFv is SEQ ID NO: 8.

6. The bispecific antibody according to claim 4, wherein the amino acid sequence of LCVR2 of each scFv is SEQ ID NO: 9.

7. The bispecific antibody according to claim 1, wherein the amino acid sequence of each HC is SEQ ID NO: 4, the amino acid sequence of each LC is SEQ ID NO: 3, the amino acid sequence of HCVR2 of each scFv is SEQ ID NO: 6 and the amino acid sequence of LCVR2 of each scFv is SEQ ID NO: 8 or SEQ ID NO: 9.

8. The bispecific antibody according to claim 7, wherein the amino acid sequence of LCVR2 of each scFv is SEQ ID NO: 8.

9. The bispecific antibody according to claim 7, wherein the amino acid sequence of LCVR2 of each scFv is SEQ ID NO: 9.

10. The bispecific antibody according to claim 7, wherein the amino acid sequence of L1 is SEQ ID NO: 23 and the amino acid sequence of L2 is SEQ ID NO: 24.

11. The bispecific antibody according to claim 1, wherein the amino acid sequence of each HC is SEQ ID NO: 4, the amino acid sequence of each LC is SEQ ID NO: 3, the amino acid sequence of HCVR2 of each scFv is SEQ ID NO: 6, the amino acid sequence of LCVR2 of each scFv is SEQ ID NO: 8, the amino acid sequence of L1 is SEQ ID NO: 23 and the amino acid sequence of L2 is SEQ ID NO: 24.

12. The bispecific antibody according to claim 1, wherein the amino acid sequence of each HC is SEQ ID NO: 4, the amino acid sequence of each LC is SEQ ID NO: 3, the amino acid sequence of HCVR2 of each scFv is SEQ ID NO: 6, the amino acid sequence of LCVR2 of each scFv is SEQ ID NO: 9, the amino acid sequence of L1 is SEQ ID NO: 23 and the amino acid sequence of L2 is SEQ ID NO: 24.

13-18. (canceled)

19. A pharmaceutical composition comprising: a bispecific antibody comprising an immunoglobulin G (IgG) antibody and two single chain variable fragments (scFv) wherein,
(a) said IgG comprises two heavy chains (HC) and two light chains (LC), each HC comprises a heavy chain variable region (HCVR1) comprising heavy chain CDRs (HCDR) 1-3 and each light chain comprises a light chain variable region (LCVR1) comprising light chain CDRs (LCDR) 1-3, wherein the amino acid sequence of HCDR1 is SEQ ID NO: 10, the amino acid sequence of HCDR2 is SEQ ID NO: 11, the amino acid sequence of HCDR3 is SEQ ID NO: 12, the amino acid sequence of LCDR1 is SEQ ID NO: 16, the amino acid sequence of LCDR2 is SEQ ID NO: 17, and the amino acid sequence of LCDR3 is SEQ ID NO: 18; and
(b) each scFv comprises a heavy chain variable region (HCVR2) and a light chain variable region (LCVR2), the HCVR2 comprising HCDRs 4-6, and the LCVR2 comprising LCDRs 4-6, wherein the amino acid sequence of HCDR4 is SEQ ID NO: 13, the amino acid sequence of HCDR5 is SEQ ID NO: 14, the amino acid sequence of HCDR6 is SEQ ID NO: 15, the amino acid sequence of LCDR4 is SEQ ID NO: 19, the amino acid sequence of LCDR5 is SEQ ID NO: 20, and the amino acid sequence of LCDR6 is SEQ ID NO: 21 or SEQ ID NO: 22,
wherein each scFv is linked at the N-terminus of HCVR2 of each scFv to said IgG antibody at the C-terminus of each IgG HC via a polypeptide linker (L1),
wherein the HCVR2 of each scFv is linked at the C-terminus of the HCVR2 to the LCVR2 of the same scFv at the N-terminus of the LCVR2 of the same scFv via a second polypeptide linker (L2)
and wherein the bispecific antibody binds to human calcitonin gene related peptide (CGRP) and the p19 subunit of human IL-23; and one or more pharmaceutically acceptable carriers, diluents or excipients.

20. A method of treating autoimmune diseases comprising administering to a patient in need thereof an effective amount of a bispecific antibody comprising an immunoglobulin G (IgG) antibody and two single chain variable fragments (scFv) wherein,
(a) said IgG comprises two heavy chains (HC) and two light chains (LC), each HC comprises a heavy chain variable region (HCVR1) comprising heavy chain CDRs (HCDR) 1-3 and each light chain comprises a light chain variable region (LCVR1) comprising light chain CDRs (LCDR) 1-3, wherein the amino acid sequence of HCDR1 is SEQ ID NO: 10, the amino acid sequence of HCDR2 is SEQ ID NO: 11, the amino acid sequence of HCDR3 is SEQ ID NO: 12, the amino acid sequence of LCDR1 is SEQ ID NO: 16, the amino acid sequence of LCDR2 is SEQ ID NO: 17, and the amino acid sequence of LCDR3 is SEQ ID NO: 18; and
(b) each scFv comprises a heavy chain variable region (HCVR2) and a light chain variable region (LCVR2), the HCVR2 comprising HCDRs 4-6, and the LCVR2 comprising LCDRs 4-6, wherein the amino acid sequence of HCDR4 is SEQ ID NO: 13, the amino acid sequence of HCDR5 is SEQ ID NO: 14, the amino acid sequence of HCDR6 is SEQ ID NO: 15, the amino acid sequence of LCDR4 is SEQ ID NO: 19, the amino acid sequence of LCDR5 is SEQ ID NO: 20, and the amino acid sequence of LCDR6 is SEQ ID NO: 21 or SEQ ID NO: 22,
wherein each scFv is linked at the N-terminus of HCVR2 of each scFv to said IgG antibody at the C-terminus of each IgG HC via a polypeptide linker (L1),
wherein the HCVR2 of each scFv is linked at the C-terminus of the HCVR2 to the LCVR2 of the same scFv at the N-terminus of the LCVR2 of the same scFv via a second polypeptide linker (L2)
and wherein the bispecific antibody binds to human calcitonin gene related peptide (CGRP) and the p19 subunit of human IL-23.

21. A method according to claim 20, wherein the autoimmune disease is one of inflammatory bowel disease, Crohn's disease, ulcerative colitis, psoriatic arthritis, and ankylosing spondylitis.

22-28. (canceled)

29. The pharmaceutical composition according to claim 19, wherein the amino acid sequence of HCVR1 of each HC of the bispecific antibody is SEQ ID NO: 5, the amino acid sequence of LCVR1 of each LC of the bispecific antibody is SEQ ID NO: 7, the amino acid sequence of HCVR2 of each scFv of the bispecific antibody is SEQ ID NO: 6 and the amino acid sequence of LCVR2 of each scFv of the bispecific antibody is SEQ ID NO: 8 or SEQ ID NO: 9.

30. The pharmaceutical composition according to claim 19, wherein the amino acid sequence of each HC of the bispecific antibody is SEQ ID NO: 4, the amino acid sequence of each LC of the bispecific antibody is SEQ ID NO: 3, the amino acid sequence of HCVR2 of each scFv of the bispecific antibody is SEQ ID NO: 6 and the amino acid sequence of LCVR2 of each scFv of the bispecific antibody is SEQ ID NO: 8 or SEQ ID NO: 9.

31. The pharmaceutical composition according to claim 19, wherein the amino acid sequence of each HC of the bispecific antibody is SEQ ID NO: 4, the amino acid sequence of each LC of the bispecific antibody is SEQ ID NO: 3, the amino acid sequence of HCVR2 of each scFv of the bispecific antibody is SEQ ID NO: 6, the amino acid sequence of LCVR2 of each scFv of the bispecific antibody is SEQ ID NO: 8, the amino acid sequence of L1 of the bispecific antibody is SEQ ID NO: 23 and the amino acid sequence of L2 of the bispecific antibody is SEQ ID NO: 24.

32. The pharmaceutical composition according to claim 19, wherein the amino acid sequence of each HC of the bispecific antibody is SEQ ID NO: 4, the amino acid sequence of each LC of the bispecific antibody is SEQ ID NO: 3, the amino acid sequence of HCVR2 of each scFv of the bispecific antibody is SEQ ID NO: 6, the amino acid sequence of LCVR2 of each scFv of the bispecific antibody is SEQ ID NO: 9, the amino acid sequence of L1 of the bispecific antibody is SEQ ID NO: 23 and the amino acid sequence of L2 of the bispecific antibody is SEQ ID NO: 24.

33. The method according to claim 21, wherein the amino acid sequence of each HC of the bispecific antibody is SEQ ID NO: 4, the amino acid sequence of each LC of the bispecific antibody is SEQ ID NO: 3, the amino acid sequence of HCVR2 of each scFv of the bispecific antibody is SEQ ID NO: 6, the amino acid sequence of LCVR2 of each scFv of the bispecific antibody is SEQ ID NO: 8, the amino acid sequence of L1 of the bispecific antibody is SEQ ID NO: 23 and the amino acid sequence of L2 of the bispecific antibody is SEQ ID NO: 24.

34. The method according to claim 21, wherein the amino acid sequence of each HC of the bispecific antibody is SEQ ID NO: 4, the amino acid sequence of each LC of the bispecific antibody is SEQ ID NO: 3, the amino acid sequence of HCVR2 of each scFv of the bispecific antibody is SEQ ID NO: 6, the amino acid sequence of LCVR2 of each scFv of the bispecific antibody is SEQ ID NO: 9, the amino acid sequence of L1 of the bispecific antibody is SEQ ID NO: 23 and the amino acid sequence of L2 of the bispecific antibody is SEQ ID NO: 24.
